(19) United States
(12) Patent Application Publication
(10) Pub. No.: US 2026/0217699 A1
(43) Pub. Date: Jul. 30, 2026

(54) BERBERINE TAUROURSODEOXYCHOLATE COMPOSITIONS AND METHODS THEREOF

(71) Applicant: SHENZHEN HIGHTIDE BIOPHARMACEUTICAL LTD., Shenzhen (CN)

(72) Inventors: Liping Liu, Shenzhen (CN); Ru Bai, Shenzhen (CN); Xinxiang Fu, Shenzhen (CN); Junwei Cheng, Shenzhen (CN); Mingzheng Xie, Shenzhen (CN)

(21) Appl. No.: 19/149,826

(22) PCT Filed: Feb. 8, 2024
(86) PCT No.: PCT/CN2024/076990

§ 371 Date: Jul. 21, 2025

Related U.S. Application Data
(60) Provisional application No. 63/446,004, filed on Feb. 15, 2023.

(30) Foreign Application Priority Data
Feb. 5, 2024 (CN) 2024101662972

(51) Int. Cl. C07D 455/03; A61K 31/4375; A61K 31/575; A61P 25/16; C07J 41/00

(52) U.S. Cl. CPC C07D 455/03; A61K 31/4375; A61K 31/575; A61P 25/16; C07J 41/0061; C07B 2200/13

(57) ABSTRACT

The invention provides berberine tauroursodeoxycholate (BTUDC), pharmaceutical compositions and methods of use thereof for the treatment, reduction and/or prevention of Parkinson's disease, or an associated disease and disorder, as monotherapy or in combination with other agents or as an adjuvant. The invention further provides pharmaceutical compositions and methods of use of berberine (BBR) and tauroursodeoxycholic acid (TUDCA) for the treatment, reduction and/or prevention of Parkinson's disease, or an associated disease and disorder, as monotherapy or in combination with other agents or as an adjuvant.

### PRIORITY CLAIMS AND RELATED PATENT APPLICATIONS

This application claims the benefit of priority to U.S. Provisional Application Ser. No. 63/446,004, filed Feb. 15, 2023, and Chinese Patent Application No. 2024101662972 filed on Feb. 6, 2024, all of which are incorporated herein by reference in their entireties.

### TECHNICAL FIELD OF THE INVENTION

The invention generally relates to pharmaceutical compositions and methods for therapeutic uses thereof. In particular, the invention relates to berberine tauroursodeoxycholate (BTUDC), pharmaceutical compositions and methods of use thereof for the treatment, reduction and/or prevention of central nervous system (CNS) diseases and disorders, such as Parkinson's disease and amyotrophic lateral sclerosis (ALS), or an associated disease and disorder, as monotherapy or in combination with other agents or as an adjuvant. The invention further relates to pharmaceutical compositions and methods of use of berberine (BBR) and tauroursodeoxycholic acid (TUDCA) for the treatment, reduction and/or prevention of Parkinson's disease, or an associated disease and disorder, as monotherapy or in combination with other agents or as an adjuvant.

### BACKGROUND OF THE INVENTION

Degenerative nerve diseases (or neurodegenerative diseases) affect millions of people worldwide. These diseases include a range of conditions that primarily affect the neurons in the brain. Neurons are building blocks of the nervous system that includes the brain and spinal cord. Neurons normally do not reproduce or replace themselves when they become damaged or die. Examples of neurodegenerative diseases affecting the CNS include Parkinson's disease, Alzheimer's disease, Huntington's disease and ALS. Currently, neurodegenerative diseases are incurable and debilitating conditions that result in progressive degeneration and/or death of nerve cells.

Parkinson's disease is a long-term degenerative disorder of the central nervous system that causes unintended or uncontrollable movements and difficulty with balance and coordination. Symptoms usually begin gradually and worsen over time. Early-stage symptoms include tremor, rigidity, slowness of movement, and difficulty with walking. As the disease progresses, people may have difficulty walking and talking. Cognitive and behavioral problems, such as depression, anxiety and apathy, may also occur in many patients. Parkinson's disease dementia becomes common in the advanced stages of the disease. Those with Parkinson's can also have problems with their sleep and sensory systems. As the disease progresses, patients may have difficulty walking and talking. (Sveinbjornsdottir 2016 “The clinical symptoms of Parkinson's disease” J. Neurochem. 139 (Suppl 1): 318-324; “Parkinson's Disease Information Page Nat'l Inst. Neurol. Dis. & Stroke, https://www.ninds.nih.gov/health-information/disorders/parkinsons-disease.)

Currently, there is no cure or effective treatment for Parkinson's disease. Treatment is usually aimed for reducing the effects of the symptoms. Initial treatment options include levodopa (L-DOPA), MAO-B inhibitors, and dopamine agonists. These medications become less effective as the disease progresses, while at the same time producing a side effect marked by involuntary muscle movements. Deep brain stimulation with surgically placed microelectrodes has been used to reduce motor symptoms in severe cases where drugs are ineffective. Diet and certain forms of rehabilitation have shown some effectiveness at improving symptoms. (Samii, et al. 2004 “Parkinson's disease” Lancet 363 (9423):1783-1793; Armstrong, et al. 2020 “Diagnosis and Treatment of Parkinson Disease: A Review” JAMA 323(6):548-560; Barichella, et al. 2009 “Major nutritional issues in the management of Parkinson's disease” Movement Disorders 24(13):1881-1892.)

Currently available therapeutics and methods for treating Parkinson's disease remain inadequate. An urgent need exists for a novel, safe and effective treatment.

### SUMMARY OF THE INVENTION

In one aspect, the invention generally relates to a salt having Formula (I):

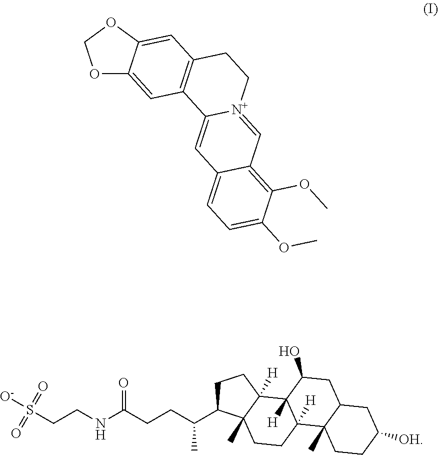

In another aspect, the invention generally relates to a solid form of a compound of Formula (I), which is Form A, wherein X-ray powder diffraction (XRPD) pattern thereof comprises one or more characteristic diffraction peaks at the following 2θ angles: 4.62°, 9.32°, 17.02°±0.2°, with a radiation source of Cu-Kα.

In another aspect, the invention generally relates to a pharmaceutical composition comprising BTUDC and a pharmaceutically acceptable excipient, carrier, or diluent.

In another aspect, the invention generally relates to a pharmaceutical composition comprising a solid form disclosed herein, and a pharmaceutically acceptable excipient, carrier, or diluent.

In yet another aspect, the invention generally relates to a unit dosage comprising a pharmaceutical composition of BTUDC.

In yet another aspect, the invention generally relates to a method for reducing, preventing or treating a neurodegenerative disease, comprising administering to a subject in need thereof a pharmaceutical composition comprising BTUDC.

In yet another aspect, the invention generally relates to a method for reducing, preventing or treating Parkinson's disease, comprising administering to a subject in need thereof a pharmaceutical composition comprising BTUDC.

In yet another aspect, the invention generally relates to a method for reducing, preventing or treating a neurodegenerative disease, comprising administering to a subject in need thereof a pharmaceutical composition comprising BBR and TUDCA.

In yet another aspect, the invention generally relates to method for reducing, preventing or treating Parkinson's disease, comprising administering to a subject in need thereof a pharmaceutical composition comprising BBR and TUDCA.

In yet another aspect, the invention generally relates to use of BTUDC for treating a neurodegenerative disease, or a related disease or disorder.

In yet another aspect, the invention generally relates to use of BTUDC for treating Parkinson's disease, or a related disease or disorder.

In yet another aspect, the invention generally relates to use of BTUDC for the manufacture of a medicament for prevention or treatment of a neurodegenerative disease, or a related disease or disorder.

In yet another aspect, the invention generally relates to use of BTUDC for the manufacture of a medicament for prevention or treatment of Parkinson's disease, or a related disease or disorder.

In yet another aspect, the invention generally relates to use of BBR and TUDCA for treating a neurodegenerative disease, or a related disease or disorder.

In yet another aspect, the invention generally relates to use of BBR and TUDCA for treating Parkinson's disease, or a related disease or disorder.

In yet another aspect, the invention generally relates to use of BBR and TUDCA for the manufacture of a medicament for prevention or treatment of a neurodegenerative disease, or a related disease or disorder.

In yet another aspect, the invention generally relates to use of BBR and TUDCA for the manufacture of a medicament for prevention or treatment of Parkinson's disease, or a related disease or disorder.

In yet another aspect, the invention generally relates to a method for making the BTUDC salt disclosed herein.

In yet another aspect, the invention generally relates to a method for preparing a solid form disclosed herein.

### BRIEF DESCRIPTION OF THE DRAWINGS

FIG. 1 shows an exemplary ¹H NMR spectrum of BTUDC.

FIG. 2 shows an exemplary ¹H NMR spectrum of TUDCA.

FIG. 3 shows an exemplary ¹H NMR spectrum of BBR-Cl.

FIG. 4 shows a schematic representation of asymmetric structural unit.

FIG. 5 shows a schematic representation of crystal single-cell structure.

FIG. 6 shows exemplary results regarding effect of Test Article for Number of Ipsilateral Rotation Within 30 minutes of apomorphine injection in 6-hydroxydopamine (6-OHDA) Stereotactic Injection Induced Rats.

FIG. 7 shows exemplary results regarding effect of Test Article on Time on the Beam Test in 6-OHDA Stereotactic Injection Induced Rats.

FIG. 8 shows exemplary results regarding effect of Test Article for Number of feet slips on the Beam test in 6-OHDA Stereotactic Injection Induced Rats.

FIG. 9 shows exemplary results regarding effect of Test Article on Time on Rotarod Test in 6-OHDA Stereotactic Injection Induced Rats.

FIG. 10 shows exemplary results regarding effect of Test Article on Peak Grip in Grip Strength Test in 6-OHDA Stereotactic Injection Induced Rats.

FIG. 11 shows exemplary XRPD pattern of Form A.

FIG. 12 shows exemplary DSC and TGA graphs of Form A.

FIG. 13 shows exemplary XRPD comparison of crystalline transition of Form A

FIG. 14 shows exemplary Fast DVS isotherm plot of Form A.

FIG. 15 shows exemplary XRPD of Form A before and after Fast DVS.

FIG. 16 shows exemplary XRPD of a Form A single crystal sample.

FIG. 17 shows exemplary XRPD patterns of Form A from stability study.

FIG. 18 shows exemplary data on the effect of test article on the number of ipsilateral rotations within 30 minutes of apomorphine injection in 6-OHDA stereotactic injection induced rats.

FIG. 19 shows exemplary data on the effect of test article on the peak grip in grip strength test in 6-OHDA stereotactic injection induced rats.

FIG. 20 shows exemplary data on the effect of test article on the time on rod in rotarod test in 6-OHDA stereotactic injection induced rats.

FIG. 21 shows exemplary data on the time to pass the balance beam.

FIG. 22 shows exemplary data on the number of feet slips on balance beam.

FIG. 23 shows exemplary data on immunofluorescence staining of tyrosine hydroxylase (TH) in striatum (Str) and substantia nigra (SN) brain regions of PD model rats after 21 days of drug administration.

FIG. 24 shows exemplary data on fluorescence intensity analysis of tyrosine hydroxylase (TH)-positive cells in the Str brain region of rats 21 days after stereotactic injection of 6-OHDA in brain.

FIG. 25 shows exemplary data on fluorescence intensity analysis of tyrosine hydroxylase (TH)-positive cells in the Str brain region of rats 21 days after stereotactic injection of 6-OHDA in brain.

FIG. 26 shows exemplary data on immunofluorescence staining results of microglia (Ibal) in Str and SN brain regions of PD model rats after 21 days of drug administration.

FIG. 27 shows exemplary data analysis of microglia (Ibal) positive cell counts in the Str brain region of rats 21 days after brain stereotactic injection of 6-OHDA.

FIG. 28 shows exemplary data analysis of microglia (Ibal) positive cell counts in the SN brain region of rats 21 days after stereotactic injection of 6-OHDA in the brain.

FIG. 29 shows exemplary data analysis of Elisa test results of 1L-1β, IL-6 and TNF-α in rat cerebrospinal fluid after 21 days of administration.

FIG. 30 shows exemplary ¹H NMR spectrum of BTUDC prepared by method 2.

FIG. 31 shows exemplary XRPD pattern of BTUDC raw material prepared by method 1.

FIG. 32 shows exemplary TGA and DSC of BTUDC raw material prepared by method 1.

FIG. 33 shows exemplary On-line Temperature Change Experiment of BTUDC raw material prepared by method 1.

### DEFINITIONS

Unless defined otherwise, all technical and scientific terms used herein have the same meaning as commonly understood by one of ordinary skill in the art to which this invention belongs. The abbreviations used herein have their conventional meaning within the chemical and biological arts. The chemical structures and formulae set forth herein are constructed according to the standard rules of chemical valency known in the chemical arts.

As used in the present disclosure, the following words and phrases are generally intended to have the meanings as set forth below unless expressly indicated otherwise or the context in which they are used indicates otherwise.

In this specification and the appended claims, the singular forms “a,” “an,” and “the” include plural reference, unless the context clearly dictates otherwise.

The term “and/or” is used in this disclosure to mean either “and” or “or” unless the context clearly dictates otherwise.

As used herein, “at least” a specific value is understood to be that value and all values greater than that value.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein can be modified by the term about.

At various places in the present specification, variables or parameters are disclosed in groups or in ranges. It is specifically intended that the description include each and every individual subcombination of the members of such groups and ranges. For example, a range of 1 to 16 is understood to include any number, combination of numbers, or sub-range from the group consisting 1, 2, 3, 4, 5, 6, 7, 8, 9, 10, 11, 12, 13, 14, 15 or 16.

As used herein, “XRPD” refers to X-ray powder diffraction. An XRPD pattern is an x-y graph with 20 (diffraction angle) plotted on the x-axis and intensity plotted on the y-axis. These are the diffraction peaks which may be used to characterize a crystalline material. The diffraction peaks are usually represented and referred to by their position on the x-axis rather than the intensity of the diffraction peaks on the y-axis because diffraction peak intensity can be particularly sensitive to sample orientation (see Pharmaceutical Analysis, Lee & Web, pp. 255-257 (2003)). Thus, intensity is not typically used by those of skill in the art to characterize a crystalline material.

As used herein, the term “2 theta value” or “2θ” refers to the peak position in degrees based on the experimental setup of the X-ray diffraction experiment and is a common abscissa unit in diffraction patterns. The experimental setup requires that if a reflection is diffracted when the incoming beam forms an angle theta (θ) with a certain lattice plane, the reflected beam is recorded at an angle 2 theta (2θ). It should be understood that reference herein to specific 2θ values for a specific solid form is intended to mean the 2θ values (in degrees) as measured using the X-ray diffraction experimental conditions as described herein.

As with any data measurement, there may be variability in XRPD data. In addition to the variability in diffraction peak intensity, there may also be variability in the position of the diffraction peaks on the x-axis. This variability can, however, typically be accounted for when reporting the positions of diffraction peaks for purposes of characterization. Such variability in the position of diffraction peaks along the x-axis may be derived from several sources. One such source can be sample preparation. Samples of the same crystalline material prepared under different conditions may yield slightly different diffractograms. Factors such as particle size, moisture content, solvent content, temperature, and orientation may all affect how a sample diffracts X-rays. Another source of variability comes from instrument parameters. Different X-ray powder diffractometers operate using different parameters and may lead to slightly different diffraction patterns from the same crystalline material. Likewise, different software packages process XRPD data differently and this may also lead to variability. These and other sources of variability are known to those of ordinary skill in the art. Due to such sources of variability, the values of each X-ray diffraction peak may be preceded with the term “about” or proceeded with an appropriate range defining the experimental variability (e.g., ±0.10, ±0.2°, ±0.3°, ±0.4°, 0.5°, etc.).

Crystalline forms, such as crystalline forms of a compound of Formula (I), are readily analyzed by XRPD. The data from X-ray powder diffraction may be used in multiple ways to characterize crystalline forms. For example, the entire x-ray powder diffraction pattern output from a diffractometer may be used to characterize a crystalline form (e.g., of a compound of Formula (I)). A smaller subset of such data, however, may also be suitable and used for characterizing such crystalline forms. Indeed, often even a single x-ray powder diffraction peak may be used to characterize such a crystalline form. With respect to crystalline forms of a compound of Formula (I), any one or more of the peaks in the x-ray powder diffraction pattern may be used to characterize the crystalline form of a compound of Formula (I) disclosed herein.

The term “characteristic peaks” when referring to the peaks in an XRPD pattern of a crystalline form of a given chemical entity (e.g., a crystalline form of a compound of formula (I)) refers to a collection of specific diffraction peaks whose values span a range of 2θ values (e.g., 0°-40°) that are, as a whole, unique to that specific crystalline form.

Differential scanning calorimetry (DSC) profiles can be particularly sensitive to sample preparation and parameters. Thus, there may be variability in DSC data (e.g., location of onset and peak maximum temperatures). Due to such sources of variability, the values of each temperature based on DSC data may be preceded with the term “about” or proceeded with an appropriate range defining the experimental variability (e.g., ±0.5° C., ±1° C., ±3° C., ±4° C., 5° C., etc.).

As used herein, the term “crystalline” refers to any solid substance exhibiting three-dimensional order, which in contrast to an amorphous solid substance, gives a distinctive X-ray powder diffraction (XRPD) pattern with sharply defined peaks.

As used herein, the term “amorphous” refers to any solid substance that lacks order in three dimensions. In some instances, amorphous solids may be characterized by known techniques, including XRPD crystallography, solid-state nuclear magnet resonance (ssNMR) spectroscopy, DSC, or some combination of these techniques. Amorphous solids give diffuse XRPD patterns, typically comprised of one or two broad peaks (i.e., peaks having base widths of about 5° 2 θ, or greater).

As used herein, the term “polymorph” refers to different crystalline forms of the same compound and includes, but is not limited to, other solid state molecular forms including hydrates (e.g., bound water present in the crystalline structure) and solvates (e.g., bound solvents other than water) of the same compound.

As used herein, the term “essentially the same” with reference to X-ray powder diffraction peak positions and/or patterns means that typical peak position and intensity variability are taken into account. For example, one skilled in the art will appreciate that the peak positions (2θ) will show some variability, typically as much as 0.1 to 0.2 degrees, as well as on the apparatus being used to measure the diffraction. Further, one skilled in the art will appreciate that relative peak intensities will show inter-apparatus variability as well as variability due to degree of crystallinity, preferred orientation, prepared sample surface, and other factors known to those skilled in the art, and should be taken as qualitative measures only. Similarly, as used herein, “essentially the same” with reference to DSC is intended to also encompass the variability associated with these analytical techniques, which are known to those of skill in the art.

As used herein, the term “stable” refers to a compound that is not substantially altered when subjected to conditions to allow for its production, detection, recovery, purification, and use for one or more of the purposes disclosed herein. In some embodiments, a stable compound or chemically feasible compound is one that is not substantially altered when kept at a temperature of 40° C. or less, in the absence of moisture or other chemically reactive conditions, for at least a week, preferably for at least a month, more preferably for at least six months, even more preferably for at least one year.

As used herein, the term “solvate” refers to a crystalline solid adduct containing either stoichiometric or nonstoichiometric amounts of a solvent incorporated within the crystal structure. When the solvent is tightly bound to the drug the resulting complex will have a well-defined stoichiometry that is independent of humidity. When, however, the solvent is weakly bound, as in channel solvates and hygroscopic compounds, the solvent content will be dependent on humidity and drying conditions. In such cases, the complex will often be non-stoichiometric. If the incorporated solvent is water, such adduct is referred to as a “hydrate”. Thus, the term “hydrate” describes a solvate comprising the drug substance and a stoichiometric or non-stoichiometric amount of water.

As used herein, the term “anhydrous” or “anhydrate” when referring to a crystalline form (e.g., a crystalline form of the compound of Formula (I)) means that no water molecules form a portion of the unit cell of the crystalline form. An anhydrous crystalline form may nonetheless contain water molecules that do not form part of the unit cell of the anhydrous crystalline form (e.g., as residual solvent molecule left behind from the production of the crystalline form). In a preferred embodiment, water can make up about 0.5% by weight of the total composition of a sample of an anhydrous form. In a more preferred embodiment, water can make up about 0.2% by weight of the total composition of a sample of an anhydrous form. In some embodiments, a sample of an anhydrous crystalline form of the compound of formula (I) contains no water molecules, e.g., no detectable amount of water.

As used herein, “pharmaceutical composition” refers to the combination of a therapeutically active agent with one or more pharmaceutically acceptable excipients, carriers, or diluents, making the composition especially suitable for diagnostic or therapeutic use in vivo or ex vivo.

As used herein, the term “pharmaceutically acceptable excipient, carrier, or diluent” refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject. A “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal.

As used herein, “administering” means oral administration, administration as a pulmonary, suppository, intramuscular administration, intrathecal administration, intranasal administration or subcutaneous administration, or the implantation of a slow-release device, e.g., a mini-osmotic pump, to a subject. Administration is by any route, including transmucosal (e.g., buccal, sublingual, palatal, gingival, nasal, vaginal, rectal, or). Parenteral administration includes, e.g., intramuscular and subcutaneous. Other modes of delivery include, but are not limited to, the use of liposomal formulations, etc. By “co-administer” it is meant that a composition described herein is administered at the same time, just prior to, or just after the administration of one or more additional therapies (e.g., therapeutic agent, chemotherapeutic, or treatment for a neurodegenerative disease). The compound of formula (I) can be administered alone or can be co-administered to the patient. Co-administration is meant to include simultaneous or sequential administration of the compound individually or in combination (more than one compound or agent). Thus, the preparations can also be combined, when desired, with other active substances (e.g., to reduce metabolic degradation).

The terms “disease,” “disorder,” and “condition” are used interchangeably herein.

As used herein, the term “treating”, “reducing”, or “preventing” a disease or disorder refers to ameliorating such a condition before or after it has occurred. As compared with an equivalent untreated control, such reduction or degree of prevention is at least 5%, 10%, 20%, 40%, 50%, 60%, 80%, 90%, 95%, or 100% as measured by any standard technique. The terms “treat,” “treating” and “treatment” contemplate an action that occurs while a subject is suffering from the specified disease, disorder or condition, which reduces the severity of the disease, disorder or condition, or retards or slows the progression of the disease, disorder or condition (“therapeutic treatment”), and also contemplates an action that occurs before a subject begins to suffer from the specified disease, disorder or condition (“prophylactic treatment”). In one embodiment, the compounds provided herein are contemplated to be used in methods of therapeutic treatment wherein the action occurs while a subject is suffering from the specified disease, disorder or condition and results in a reduction in the severity of the disease, disorder or condition, or retardation or slowing of the progression of the disease, disorder or condition. In an alternate embodiment, the compounds provided herein are contemplated to be used in methods of prophylactic treatment wherein the action occurs before a subject begins to suffer from the specified disease, disorder or condition and results in preventing a disease, disorder or condition, or one or more symptoms associated with the disease, disorder or condition, or preventing the recurrence of the disease, disorder or condition.

As used herein, the term “effective amount” of an active agent refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the patient.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

### DETAILED DESCRIPTION OF THE INVENTION

The invention is based in part on compositions and methods of use of BTUDC, as well as combinations of BBR and TUDCA, for treatment of neurodegenerative diseases, in particular Parkinson's disease.

Berberine (5,6-dihydro-9,10-dimethoxybenzo[g]-1,3-benzodioxolo[5,6-a]quinolizinium), an isoquinoline alkaloid isolated from plants such as Rhizoma Coptidis, has had a long history of medicinal use in China to treat various gastrointestinal diseases.

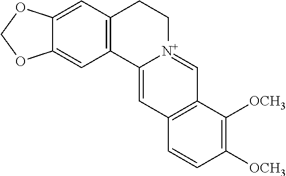 [SMILES: COc1ccc2cc3[n+](cc2c1OC)CCc1cc2c(cc1-3)OCO2]

### Berberine

Berberine is found in a variety of plants as Berberis, Hydrastis canadensis, Xanthorhiza simplicissima, Phellodendron amurense, Coptis chinensis, Tinospora cordifolia, Argemone mexicana, and Eschscholzia californica. BBR has been used for digestive ailments including travelers diarrhea. BBR has broad-spectrum activities with multiple modes of action. Previous studies on berberine reported that it exhibits antiviral, anti-inflammatory, hepatoprotective benefits, as well as in reduction of oxidative stress. For example, berberine has shown antiviral activities such as anti-influenza, anti-hepatitis C, anticytomegalovirus, and anti-alphavirus. (Neag, et al. 2018 “Berberine: Botanical Occurrence, Traditional Uses, Extraction Methods, and Relevance in Cardiovascular, Metabolic, Hepatic, and Renal Disorders” Front. Pharmacol., 21 August Sec. Ethnopharmacology vol. 9; “Berberine” Altern. Med. Rev. 2000 Apr. 5(2):175-7.)

Berberine can be obtained commercially in the form of chloride, sulfate or tannate salt, with berberine hydrochloride having been used in almost all previous studies. The low bioavailability of berberine in the current available forms makes its applications for the treatment of chronic and systemic disease very challenging.

Tauroursodeoxycholic acid (TUDCA), a.k.a. ursodoxicoltaurine or taurursodiol, is a naturally occurring hydrophilic bile acid.

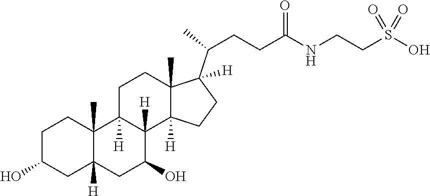

Tauroursodeoxycholic acid

TUDCA has been used for treatment of chronic cholestatic liver diseases and for gallstone. Studies have shown that TUDCA can inhibit apoptosis in different types of cells by stabilizing the mitochondrial membrane or modulating the expression of specific upstream targets of apoptosis. It has recently been reported that, in a cellular model of superoxide dismutase 1 neurodegeneration, glycine-conjugated TUDCA inhibits nitrite production and prevents matrix metallopeptidase 9 activation. Studies were conducted to collect preliminary safety and efficacy data regarding the long-term biological effects of TUDCA in patients with treated ALS. (Hofmann 1999 “The continuing importance of bile acids in liver and intestinal disease” Arch. Intern. Med. 159:2647-2658; Rodrigues, et al. 2001 “The therapeutic effects of ursodeoxycholic acid as an anti-apoptotic agent” Expert Opin. Investig. Drugs 10:1243-1253; Vaz, et al. “Glycoursodeoxycholic acid reduces matrix metalloproteinase-9 and caspase-9 activation in a cellular model of superoxide dismutase-1 neurodegeneration” Mol. Neurobiol. 2014; Elia, et al. 2016 “Tauroursodeoxycholic acid in the treatment of patients with amyotrophic lateral sclerosis” Eur. J. Neurol. 23(1):45-52.)

BTUDC, first discovered by the present applicant and disclosed herein, is an ionic salt of BBR and TUDCA represented by:

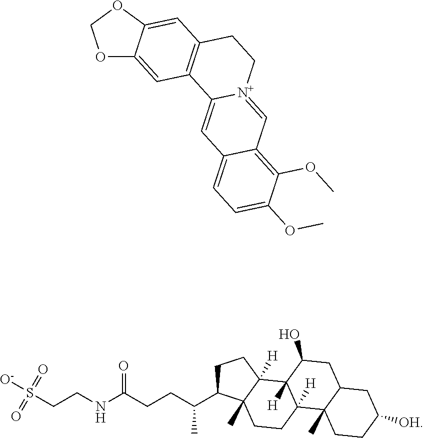

Berberine tauroursodeoxycholate (BTUDC)

Not wishing to be bound by the theories, BTUDC, as well as combinations of BBR and TUDCA, can reduce or ameliorate one or more symptoms of neurodegenerative disease, in particular Parkinson's disease. In addition, it is believed that BTUDC, as well as combinations of BBR and TUDCA, can effectively reduce, delay and/or stop the progression of Parkinson's disease.

More particularly, the present invention relates to BTUDC that synergistically combines the beneficial effects of TUDCA and BBR. The present invention thus provides a unique approach for treatment for neurodegenerative disease, in particular Parkinson's disease, alone or in combination with other available treatment or therapies. Accordingly, the present invention provides a novel strategy for treatment of neurodegenerative diseases, in particular Parkinson's disease.

In one aspect, the invention generally relates to a salt having Formula (I):

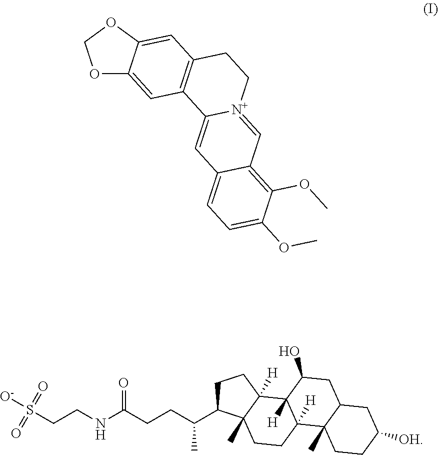

In certain embodiments, the BTUIDC salt in a substantially pure form.

In certain embodiments, the BTUDC salt is characterized by a purity of about 90% (e.g., about 95%, about 98%, about 99%) or greater.

In certain embodiments, the BTUDC salt is made by an acid-base reaction between BBR (or a salt thereof) and TUDCA (or a salt thereof).

In another aspect, the invention generally relates to a solid form of a compound of Formula (I), which is Form A, wherein X-ray powder diffraction (XRPD) pattern thereof comprises one or more characteristic diffraction peaks at the following 2θ angles: 4.62°, 9.32°, 17.02°±0.2°, with a radiation source of Cu-Kα.

In certain embodiments, the XRPD pattern of the solid form further comprises one or more characteristic diffraction peaks at the following 2θ angles: 5.96°, 6.23°, 15.19°+0.2°, with a radiation source of Cu-Kα.

In certain embodiments, the XRPD pattern of the solid form comprises characteristic diffraction peaks at the following 2θ angles: 4.62°, 5.96°, 6.23°, 9.32°, 15.19°, 17.02°+0.2°, with a radiation source of Cu-Kα.

In certain embodiments, the XRPD pattern of the solid form further comprises one or more of characteristic diffraction peaks at the following 2θ angles: 11.99°, 12.56°, 12.90°+0.2°, with a radiation source of Cu-Kα.

In certain embodiments, the XRPD pattern of the solid form comprises characteristic diffraction peaks at the following 2θ angles: 4.62°, 5.96°, 6.23°, 9.32°, 11.99°, 12.56°, 12.90°, 13.32°, 14.25°, 14.88°, 15.19°, 17.02°, 17.51°, 17.73°, 18.02°,21.39°, 24.25°, 24.71°±0.2°, with a radiation source of Cu-Kα.

In certain embodiments, the XRPD pattern of the solid form comprises characteristic diffraction peaks at the following 2θ angles:

TABLE 1
2θ angle values of XRPD pattern

| Peak No. | 2θ angle (°) | d Value (Å) | Rel. Intensity |
|---|---|---|---|
| 1 | 4.62 | 19.10874 | 44.2 |
| 2 | 5.96 | 14.82808 | 35.9 |
| 3 | 6.23 | 14.20498 | 34.0 |
| 4 | 8.68 | 10.20645 | 10.4 |
| 5 | 9.32 | 9.50785 | 36.6 |
| 6 | 11.99 | 7.41569 | 12.1 |
| 7 | 12.56 | 7.08720 | 19.2 |
| 8 | 12.90 | 6.90265 | 8.7 |
| 9 | 13.32 | 6.68861 | 22.7 |
| 10 | 14.25 | 6.25919 | 8.7 |
| 11 | 14.88 | 5.99967 | 13.3 |
| 12 | 15.19 | 5.87808 | 24.6 |
| 13 | 17.02 | 5.26350 | 100.0 |
| 14 | 17.51 | 5.12181 | 21.7 |
| 15 | 17.73 | 5.05935 | 19.8 |
| 16 | 18.02 | 4.98087 | 12.8 |
| 17 | 18.54 | 4.84454 | 9.5 |
| 18 | 18.92 | 4.75052 | 5.0 |
| 19 | 19.45 | 4.62700 | 6.1 |
| 20 | 20.09 | 4.48466 | 4.8 |
| 21 | 20.97 | 4.30449 | 5.8 |
| 22 | 21.39 | 4.22380 | 14.5 |
| 23 | 21.59 | 4.18708 | 8.0 |
| 24 | 22.32 | 4.05588 | 9.4 |
| 25 | 22.49 | 4.02668 | 7.6 |
| 26 | 22.85 | 3.96748 | 9.2 |
| 27 | 23.55 | 3.85660 | 4.7 |
| 28 | 24.25 | 3.75038 | 4.9 |
| 29 | 24.71 | 3.68490 | 9.7 |
| 30 | 25.42 | 3.58868 | 6.6 |
| 31 | 26.21 | 3.48811 | 4.9 |
| 32 | 26.54 | 3.44804 | 4.2 |
| 33 | 26.96 | 3.39824 | 4.0 |
| 34 | 27.44 | 3.34263 | 3.9 |
| 35 | 28.22 | 3.25811 | 4.0 |
| 36 | 29.65 | 3.11414 | 2.7 |
| 37 | 30.86 | 3.00357 | 3.5 |
| 38 | 31.83 | 2.92109 | 3.3 |
| 39 | 32.45 | 2.87144 | 2.8 |
| 40 | 33.37 | 2.80112 | 3.0 |
| 41 | 34.21 | 2.74039 | 2.8 |
| 42 | 36.58 | 2.58493 | 2.5 |
| 43 | 40.89 | 2.35345 | 2.5 |

In certain embodiments, the XRPD pattern of the solid form is essentially the same as that shown in FIG. 11, with a radiation source of Cu-Kα.

In certain embodiments, a differential scanning calorimetry (DSC) curve of Form A comprises an endothermic peak with a peak value at about 280° C.

In certain embodiments, a thermogravimetric analysis (TGA) curve of Form A comprises a weight loss of about 0.5% to about 3% from room temperature to about 150° C.

In certain embodiments, the solid form is an anhydrate.

In certain embodiments, the solid form is a hydrate having Formula (II). In certain embodiments, the solid form is hydrate with up to 2 H₂O molecules per BTUDC molecule (i.e., BTUDC:H₂O=1: x, wherein x is in a number in the range of 0 to 2, preferably in the range of 0.3 to 1.5, further preferably in the range of 0.5 to 1.2).

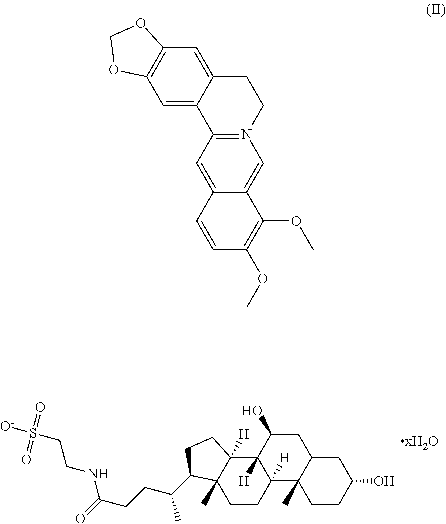

In certain embodiments, the solid form comprises hydrate and anhydrate.

In certain embodiments, the solid form is a crystalline form.

In another aspect, the invention generally relates to a pharmaceutical composition comprising BTUDC and a pharmaceutically acceptable excipient, carrier, or diluent.

In another aspect, the invention generally relates to a pharmaceutical composition comprising a solid form disclosed herein, and a pharmaceutically acceptable excipient, carrier, or diluent.

In yet another aspect, the invention generally relates to a unit dosage for comprising a pharmaceutical composition of BTUDC disclosed herein.

In yet another aspect, the invention generally relates to a method for reducing, preventing or treating a neurodegenerative disease, comprising administering to a subject in need thereof a pharmaceutical composition comprising BTUDC.

In yet another aspect, the invention generally relates to a method for reducing, preventing or treating Parkinson's disease, comprising administering to a subject in need thereof a pharmaceutical composition comprising BTUDC.

In yet another aspect, the invention generally relates to a method for reducing, preventing or treating a neurodegenerative disease, comprising administering to a subject in need thereof a pharmaceutical composition comprising BBR and TUDCA.

In yet another aspect, the invention generally relates to method for reducing, preventing or treating Parkinson's disease, comprising administering to a subject in need thereof a pharmaceutical composition comprising BBR and TUDCA.

In certain embodiments, the method results in reduction or amelioration of one or more symptoms of Parkinson's disease.

In certain embodiments, the method results in improving the effect of substantia nigra lesions.

In certain embodiments, the method results in improving chronic neroinflammation.

In certain embodiments, the method results in delay of progression of Parkinson's disease.

In certain embodiments, the method results in stop of progression of Parkinson's disease.

In certain embodiments, the method results in reversion of progression of Parkinson's disease.

In certain embodiments, the method further comprises administering to the subject a second therapeutic agent.

In certain embodiments, the second therapeutic agent is selected from levodopa (L-DOPA), MAO-B inhibitors and dopamine agonists.

In yet another aspect, the invention generally relates to use of BTUDC for treating a neurodegenerative disease, or a related disease or disorder.

In yet another aspect, the invention generally relates to use of BTUDC for treating Parkinson's disease, or a related disease or disorder.

In yet another aspect, the invention generally relates to use of BTUDC for the manufacture of a medicament for prevention or treatment of a neurodegenerative disease, or a related disease or disorder.

In yet another aspect, the invention generally relates to use of BTUDC for the manufacture of a medicament for prevention or treatment of Parkinson's disease, or a related disease or disorder.

In yet another aspect, the invention generally relates to use of BBR and TUDCA for treating a neurodegenerative disease, or a related disease or disorder.

In yet another aspect, the invention generally relates to use of BBR and TUDCA for treating Parkinson's disease, or a related disease or disorder.

In yet another aspect, the invention generally relates to use of BBR and TUDCA for the manufacture of a medicament for prevention or treatment of a neurodegenerative disease, or a related disease or disorder.

In yet another aspect, the invention generally relates to use of BBR and TUDCA for the manufacture of a medicament for prevention or treatment of Parkinson's disease, or a related disease or disorder.

In certain embodiments of the methods disclosed herein, BTUDC is administered at a daily dosage in the range of about 25 mg to about 3,500 mg for a time period of about 1 week to about 2 years.

In certain embodiments of the methods disclosed herein, the total amount of BBR and TUDCA are administered at a daily dosage in the range of about 25 mg to about 3,500 mg for a time period of about 1 week to about 2 years.

In certain embodiments, the weight ratio of BBR to TUDCA is in the range of about 10:1 to about 1:10.

In yet another aspect, the invention generally relates to a method for making the BTUDC salt disclosed herein, comprising: dissolving tauroursodeoxycholic acid in ethanol; adding an aqueous solution of NaHCO₃ to obtain a sodium tauroursodeoxycholic acid solution; dissolving berberine hydrochloride in hot water to obtain a berberine hydrochloride solution; adding the berberine hydrochloride solution dropwise into the sodium tauroursodeoxycholic acid solution; stirring the combined solution at about 60° C. to about 80° C.; and cooling the combined solution to obtain berberine tauroursodeoxycholate. In certain embodiments, the method further comprises crystalizing berberine tauroursodeoxycholate.

In yet another aspect, the invention generally relates to a method for preparing a solid form disclosed herein. The method comprises: adding sodium tauroursodeoxycholate aqueous solution to berberine chloride aqueous solution to form a combined solution; mixing the combined solution; and cooling the mixed combined solution to obtain the solid form.

In yet another aspect, the invention generally relates to a method for preparing a solid form disclosed herein. The method comprises: adding tauroursodeoxycholic acid aqueous solution to berberine chloride aqueous solution to form a combined solution; mixing the combined solution; and cooling the mixed combined solution to obtain the solid form.

Possible formulations include those suitable for oral, sublingual, buccal, parenteral (for example, subcutaneous, intramuscular, or intravenous), rectal, topical including transdermal, intranasal and inhalation administration. Most suitable means of administration for a particular patient will depend on the nature and severity of the disease or condition being treated or the nature of the therapy being used and on the nature of the active compound.

### Examples

### Characterization Methods

### Nuclear Magnetic Resonance (NMR)

NMR characterization was conducted on a Bruker AVANCE NEO 400 (Bruker, GER). Samples were prepared by dissolving 3-5 mg of the solid in Methanol-d4.

### X-ray Powder Diffraction (XRPD)

The standard XRPD patterns were collected using a Panalytical EMPYREAN (PANalytical, UK). The X-ray source was a Cu tube that was operated at 45 kV and 40 mA. Powder samples were prepared on zero-background Si holders using manual light pressure to keep the sample surface flat. Each sample was analyzed from 3 to 45° (2θ) with an effective step size of 0.013° 20. The measurement time of each sample was 3.5 min.

The form conversion by heating was carried out using an online variable temperature XRPD Malvern PANalytical Aeris (Malvern Panalytical, UK). The X-ray source was a Cu tube that was operated at 40 kV and 7.5 mA. Powder samples were prepared on zero-background Si holders using manual light pressure to keep the sample surface flat. Each sample was analyzed from 3 to 40° (2θ) with an effective step size of 0.02° 20. The measurement time of each sample was 13 min. The sample was placed on a BTS500 hot stage (Anton Paar, AT) to collected XRPD at RT, heated to the target temperature at 20° C./min and held for 10 min before XRPD analysis. The sample was then cooled to RT and characterized by XRPD.

### Thermogravimetric Analysis (TGA)

Thermogravimetric analysis (TGA) was performed on a TA Instruments Discovery 550 (TA, US). Each sample was placed into a pre-tared platinum pan and heated from RT to the set temperature at a heating rate of 10° C./min under a nitrogen atmosphere. The nitrogen purge was 40 mL/min at the balance and 60 mL/min at the furnace.

### Differential Scanning Calorimetry (DSC)

DSC analysis was conducted on a TA Instruments Discovery 250 (TA, US). Calibration of the instrument temperature and cell constant was performed using indium. The DSC cell was kept under a nitrogen purge of 50 mL/min during each analysis. The sample was placed on a Tzero hermetic pan with a pinhole and was heated from 25° C. to the set temperature at a rate of 10° C./min.

### Polarized Light Microscopy (PLM)

The polarized microscopic images of crystals were captured on a Nikon Ci-POL445 Polarizing Microscope (Nikon, JP) under a suitable objective. Oil was used for observation in some samples.

### Single-crystal X-ray Diffractometry (SCXRD)

SCXRD were collected using a XtaLAB Synergy R, D W system, HyPix. The X-ray source is a Cu Kα (λ=1.54184 Å). A suitable single crystal was selected and mounted on a glass fiber. The crystal was kept at a steady temperature at 296 K during data collection. Preliminary examination and data collection were performed and analyzed with the CrysAlisPro software package.

Cell parameters and an orientation matrix for data collection were retrieved and refined by CrysAlisPro using the setting angles of 33656 reflections in the range 2.32°<θ<76.01°. The final data completeness was 99.95% (θ=66.97°).

### Data Reduction

Frames were integrated with CrysAlisPro 1.171.42.84a (Rigaku Oxford Diffraction, 2023). A total of 56856 reflections were collected, of which 8736 were unique. A multi-scan absorption correction was performed using spherical harmonics as implemented in SCALE3 ABSPACK scaling algorithm. The absorption coefficient μ of this material is 1.201 mm⁻¹ and the minimum and maximum transmissions are 0.7952 and 0.8893. The Rint value was 5.26% based on intensity.

### Single Crystal Structure Solution and Refinement

The structure was solved in the space group P2₁ with the SHELXS-97 (Sheldrick, 1990) structure solution program with intrinsic phasing method and by using Olex2 as the graphical interface. The model was refined with version of SHELXS-97 (Sheldrick, 1990) using full matrix least squares on F² minimization. All non-hydrogen atoms were refined anisotropically. The positions of all hydrogen atoms were calculated geometrically and refined using the riding model.

### Single Crystal Structure Diagrams

The crystal structure representations and the thermal ellipsoids drawings were generated by Olex2.

### Example 1. Synthesis (Method 1) and Characterization of BTUDC

Dissolved tauroursodeoxycholic acid (1 equiv.) in ethanol, added aqueous solution of NaHCO₃ (0.95-1.5 equiv.), stirred for 15-60 minutes for reaction, and a sodium tauroursodeoxycholic acid solution was obtained.

Dissolved berberine hydrochloride in hot water. Added the berberine hydrochloride solution dropwise into the sodium tauroursodeoxycholic acid solution at 60-80° C., and stirred for not less than 10 mins for reaction at 60-80° C. Cooled down the solution. Filtered and dried the precipitate, and berberine tauroursodeoxycholate was obtained.

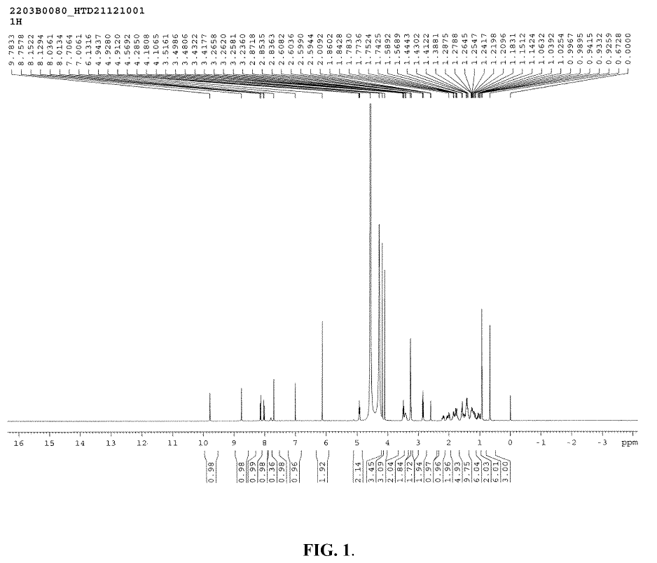

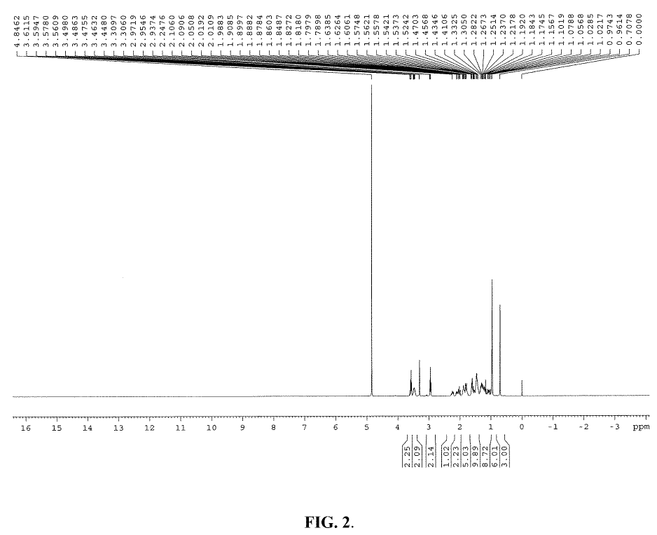

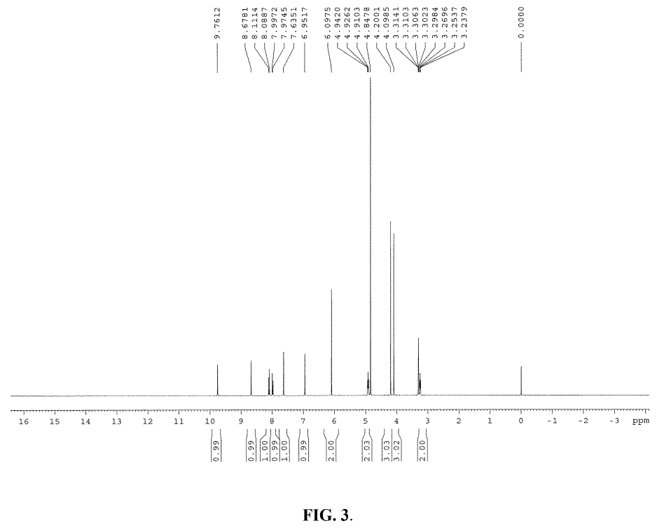

An exemplary ¹H NMR spectrum of the resulting BTUDC is provided in FIG. 1. For comparison, ¹H NMR spectra of TUDCA and BBR chloride are provided in FIG. 2 and FIG. 3, respectively.

### Single Crystal Growth and Structure Confirmation

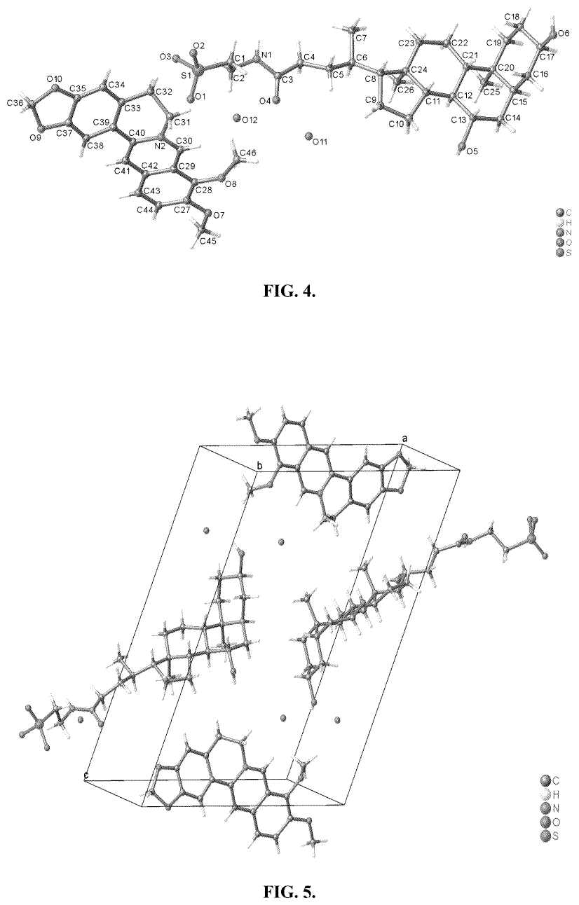

The obtained BTUDC sample was used as raw material, added to methanol/water (1/1, v/v), stirred until the solid completely dissolved and filtered, and the resulting clarified solution was allowed to stand openly at room temperature until volatilization to obtain a solid. PLM images showed the solid is clustered and needle-like crystals. The resulting needle-like crystals were tested by single-crystal X-ray diffractometry (SCXRD) and the diffraction data were analyzed to obtain the crystal structure. The crystallographic data and refinement parameters of the crystals are detailed in Table 2. A schematic representation of the asymmetric structural unit is provided in FIG. 4. FIG. 5 shows a schematic representation of the unit cell of BTUDC single crystal.

TABLE 2
Crystallographic Data and Refinement Parameters

| Empirical formula | C₄₆H₆₂N₂O₁₀S•0.82 H₂O* |
|---|---|
| Formula weight | 848.24** |
| Temperature | 296(2) K |
| Wavelength | Cu Kα (λ = 1.54184 Å) |
| Crystal system, space group | Monoclinic, P2₁ |
| Unit cell dimensions | a = 15.42465(15) Å |
| | b = 7.35920(6) Å |
| | c = 19.9879(2) Å |
| | α = 90° |
| | β = 109.5641(12)° |
| | γ = 90° |
| Volume | 2137.90(4) Å³ |
| Z, Calculated density | 2, 1.318 g/cm³ |
| Absorption coefficient | 1.196 mm⁻¹ |
| F(000) | 909.0 |
| Crystal size | 0.2 × 0.15 × 0.1 mm³ |
| 2 Theta range for data collection | 4.7° to 154.92° |
| Limiting indices | −19 ≤ h ≤ 19 |
| | −9 ≤ k ≤ 9 |
| | −24 ≤ l ≤ 22 |
| Reflections collected/ | 56856/8736 [Rᵢₙₜ = 0.0526] |
| Independent reflections | |
| Refinement method | Full-matrix least-squares |
| | on F² |
| Data/restraints/parameters | 8736/1/589 |
| Goodness-of-fit on F² | 1.044 |
| Final R indices [I ≥ 2sigma(I)] | R1 = 0.0419, wR2 = 0.1186 |
| Final R indices [all data] | R1 = 0.0438, wR2 = 0.1205 |
| Largest diff. peak and hole | 0.34/−0.20 e · Å⁻³ |
| Flack | −0.009(15) |

*No adding H for O11 and O12.
**Molecular weight with no adding H for O11 [symbol] O12

The results indicate that the crystal belongs to the monoclinic crystal system, P21 space group, with cell parameters {a=15.42465(15) Å, b=7.35920(6) Å, c=19.9879(2) Å, α=90°, β=109.5641(12)°, γ=90°,V=2137.90(4) Å3}.The single crystal structure shows that the asymmetric structural unit of this crystal structure contains one salt-forming 1:1 berberine salt of tauroursodeoxycholic acid and two water molecules, and the occupancy of the two water molecules 0.45 (011) and 0.38 (012), respectively; the chemical structural formula of this crystal is shown below. According to the analytical results, the hydrate is a non-fixed stoichiometric ratio hydrate, and the number of water molecules can vary within a certain range, e.g., within the range of 0 to 2.

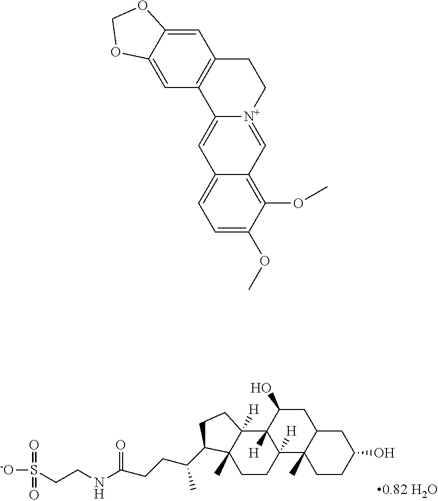

Chemical formulas of Crystalline BTUDCA

### Example 2. Synthesis (Method 2) and Characterization of BTUDC

Added tauroursodeoxycholic acid (1 equiv.) in water, stirred to dissolve. Added aqueous solution of NaHCO₃ (0.95-1.5 equiv.), stirred for at least 10 minutes, then a sodium tauroursodeoxycholate solution was obtained.

Dissolved berberine hydrochloride in hot water. Added the sodium tauroursodeoxycholate solution at 60-80° C., and stirred for not less than 10 mins for reaction at 60-80° C. Cooled down the solution. Filtered the mixture. The filter cake was washed and dried, and berberine tauroursodeoxycholate was obtained.

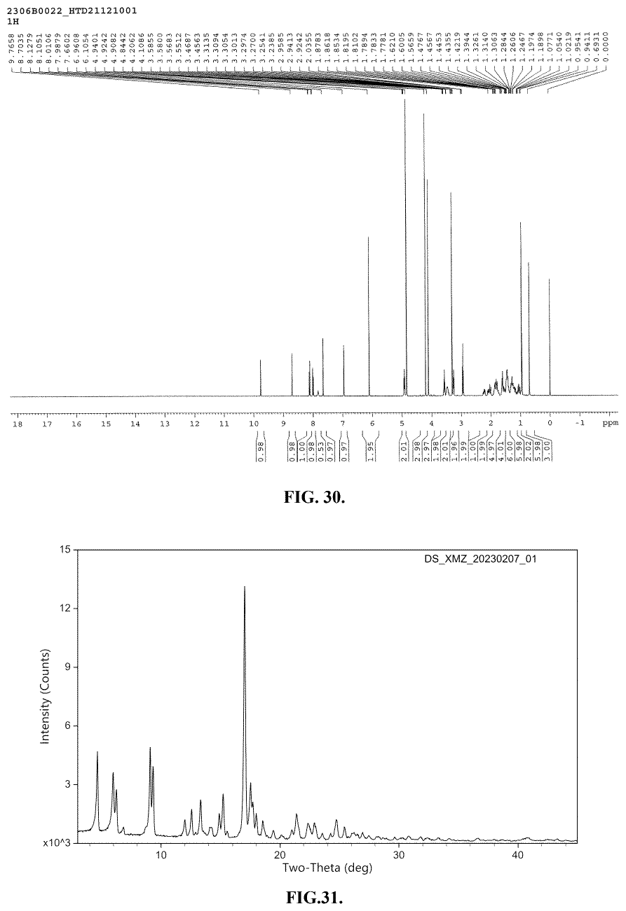

An exemplary ¹H NMR spectrum of the resulting BTUDC is provided in FIG. 30.

### Crystal Form Characterization

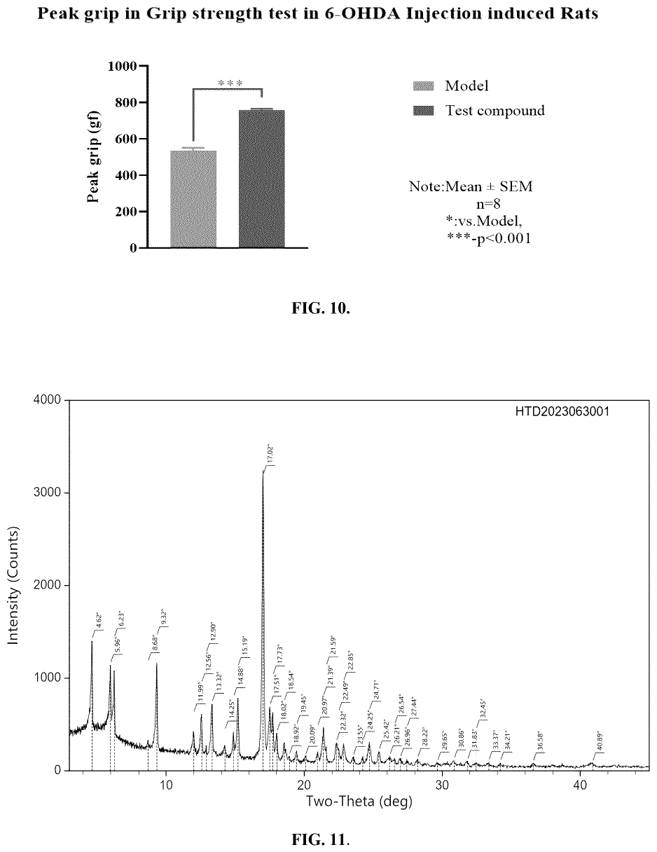

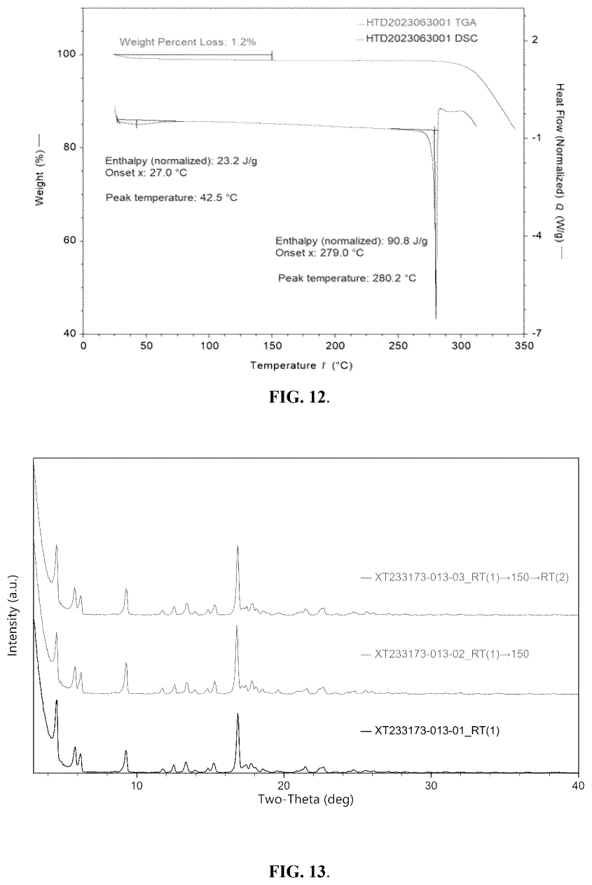

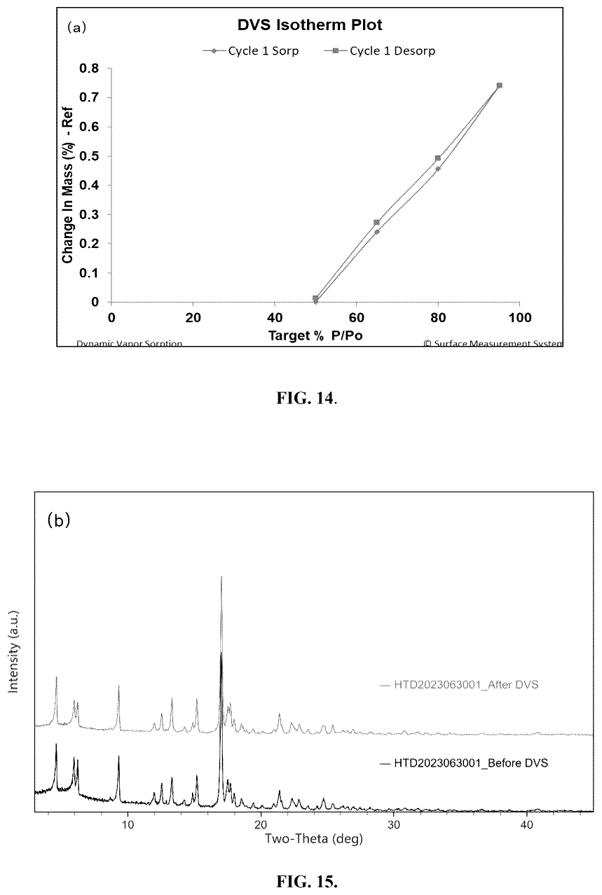

The synthesized BTUDC was designated as crystalline form A (Form A), and its XRPD diffraction and other related characterization data are shown in FIGS. 11-15. PLM images showed that Form A was a short rod-shaped particle, with a particle size generally less than 10 m; XRPD results showed that it was a crystalline solid. TGA results showed that there was a weight loss of 1.2% during the process of heating up to 150° C., and decomposition may occur above 300° C. DSC results showed that there was a wide endothermic signal from room temperature to 80° C. (peak at 43° C.), and an endothermic peak at about 280° C. Thermal transcrystallization experiments showed that the crystalline form remained unchanged during heating to 150° C. and back to room temperature, and NMR results showed that the sample was in agreement with the reference pattern. Rapid dynamic vapor sorption (DVS) results showed an adsorption weight gain of about 0.74% at 95% RH, an adsorption weight gain of about 0.46% and a desorption weight gain of about 0.49% at 80% RH. XRPD results showed that the samples did not undergo any change in crystalline shape after the DVS test compared to the pre-test. the KF titration results showed that the batch of samples had a moisture content of 2.4%.

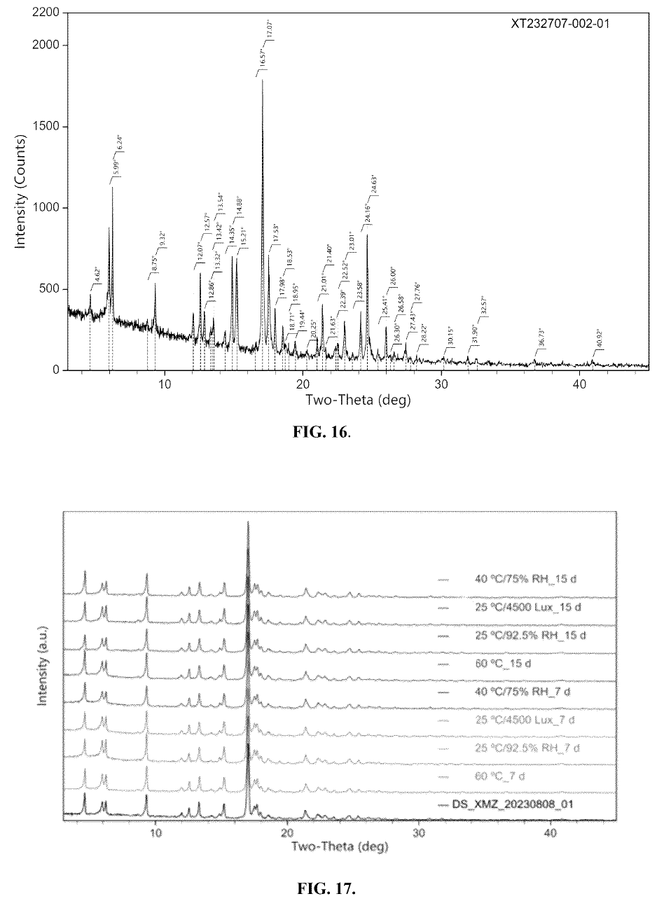

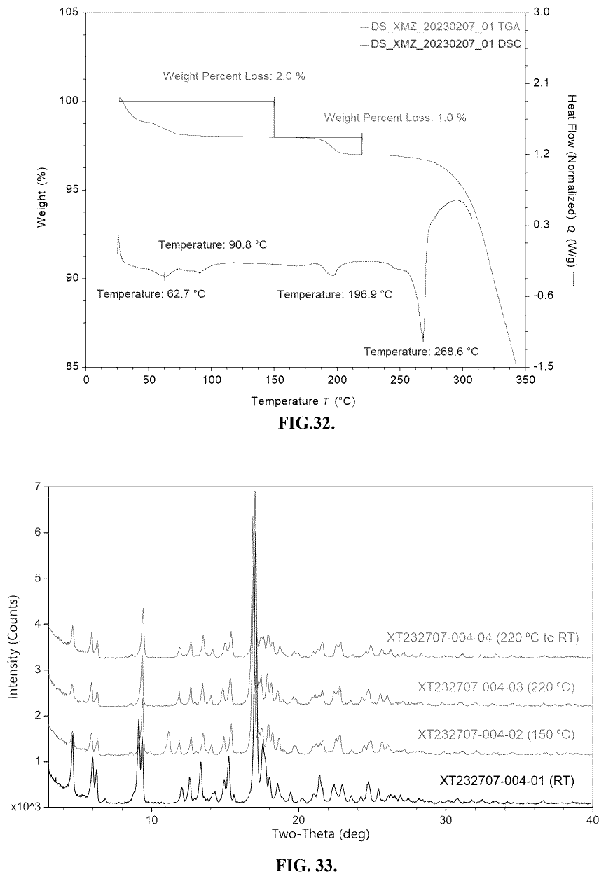

XRPD characterization of the BTUDC raw material and the single crystal sample obtained from Example 1 was performed and the results are shown in FIG. 31 and FIG. 16, respectively. Based on the comparison of the XRPD results, it can be seen that the single-crystal sample of Example 1 was of the same crystalline type as Form A. Therefore, from the characterization results of Form A and combined with the structural analysis of the single-crystal structure of BTUDC, it was determined that Form A was a hydrate with a non-fixed stoichiometric ratio. Other related characterization data for the BTUDC raw material obtained from Example 1 are shown in FIGS. 32-33.

The stability of Form A was studied under high temperature (60° C.), high humidity (25° C./92.5% RH), light (25° C./4500 Lux), and accelerated (40° C./75% RH) conditions, and the samples were taken at 7 and 15 days for XRPD characterization and HPLC tests, respectively, and the results are shown in Tables 3-4 and FIG. 17. XRPD results showed that Form A was stable under high temperature, high humidity, light and accelerated conditions for 15 days without crystalline transformation. HPLC results showed that there was no significant change in the chemical purity of Form A when it was placed under high temperature, high humidity and accelerated conditions for 15 days; and there was a significant decrease in the chemical purity of Form A when it was placed under light conditions for 15 days.

TABLE 3
Stability Study of Form A

| | | 7 Days | | 15 Days | |
|---|---|---|---|---|---|
| Ccrystalline | | XRPD | | XRPD | |
| Form | Conditions | Changes | Purity/% | Changes | Purity/% |
| Form A | High Temp. | No | 99.28 | No | 99.24 |
| (Purity: | 60° C. | Change | | Change | |
| 99.45%) | High Humidity | No | 99.44 | No | 99.44 |
| | 25° C./92.5% RH | Change | | Change | |
| | Light Exposure | No | 98.92 | No | 98.53 |
| | 25° C./4500 Lux | Change | | Change | |
| | Accelerated | No | 99.44 | No | 99.41 |
| | 40° C./75% RH | Change | | Change | |

TABLE 4
HPLC Analysis for Stability Study of Form A

| | | High | High | Light | Accel- |
|---|---|---|---|---|---|
| | | Temp. | Humidity | Exposure | erated |
| | Day 0 | 15 Days | 15 Days | 15 Days | 15 Days |
| RRT | Relative Peak Area (%) | | | | |
| 0.39 | / | / | / | 0.05 | / |
| 0.55 | / | / | / | 0.06 | / |
| 0.70 | 0.05 | 0.10 | 0.07 | 0.77 | 0.07 |
| 0.73 | / | / | / | 0.03 | / |
| 0.76 | / | / | / | 0.07 | / |
| 0.79 | / | / | / | 0.05 | / |
| 0.82 | 0.13 | 0.13 | 0.13 | 0.11 | 0.13 |
| 0.84 | 0.06 | 0.06 | 0.06 | 0.06 | 0.07 |
| 0.90 | 0.31 | 0.48 | 0.30 | 0.27 | 0.32 |
| 1.00 | 99.45 | 99.24 | 99.44 | 98.53 | 99.41 |

TABLE 5
Information regarding Raw Materials

| | | | | | |
|---|---|---|---|---|---|
| Raw | | | | | |
| Materials | Source | Batch Number | Properties | Content/Purity | Preservation |

| BTUDC* | Shenzhen | DS-XMZ- | Yellow powder | NA | Room |
|---|---|---|---|---|---|
| | High Tide | 20221215-01 | | | temperature |
| Madopar | Shanghai Roche | YT0105 | Pink tablet | NA | Protect |
| | Pharmaceuticals | | | | from light, |
| | | | | | sealed, |
| | | | | | cool and |
| | | | | | dry place |
| Sodium carboxymethyl | Xuzhou Miles | 20191203 | NA | NA | Room |
| cellulose | Biotechnology | | | | temperature |
| (CMC-Na) | | | | | |
| 6-OHDA | MedChemExpress | 154844 | Light brown to | 99.85% | 4° C. |
| | | | gray (Solid) | | |
| Apomorphine | Beijing | 100839- | White to light | 98.8% | Room |
| hydrochloride | InnoChem | 201803 | gray powder | | temperature |
| | | | | | and light |
| | | | | | protection |
| Ascorbic acid | Xuzhou Ziyang | 20201021 | White | >99.7% | Room |
| | Medical | | crystalline or | | temperature |
| | Equipment | | crystalline | | and light |
| | | | powder | | protection |

### Formulation Information

Preparation of 0.5% sodium carboxymethylcellulose (0.5% CMC-Na): Transferred 0.5g of CMC-Na to purified water, stirred till a clear solution was obtained, added water to the final volume of 100 mL.

TABLE 6
Information of Test Article

| | Concen- | | | |
|---|---|---|---|---|
| Test Article | tration | Solvent | Container | Preparation |
| BTUDC | 10 mg/mL | 0.5% CMC-Na | PE pipe | Ready-to-use |
| | 50 mg/mL | 0.5% CMC-Na | PE pipe | Ready-to-use |
| | 100 mg/mL | 0.5% CMC-Na | PE pipe | Ready-to-use |
| Madopar | 5 mg/mL | Saline | Ground and | Ready-to-use |
| | | | placed in EP | |

Preparation of PD modeling agent: 20 g 6-hydroxydopamine (6-OHDA) was dissolved in 8 μL of 0.9% Normal Saline (containing 0.02% ascorbic acid).

### Animal Model

The rats were respiratory anesthetized with isoflurane; the head was fixed to ensure that there was no movement and the brain surface was adjusted to be flat; the head was shaved, and a skin incision was made along the sagittal suture to expose the fontanel point; the glass electrode was positioned to the fontanel, and the axes of the coordinate monitor were zeroed to locate the brain areas of the SN and the Str based on the coordinates, in which the SN: AP=−5.0 mm; ML=−1.9 mm; DV=−8.5 mm, and Str: AP=+0.5 mm; ML=−3.0 mm; DV=−6.0 mm. (8.5 mm, Str: AP=+0.5 mm; ML=−3.0 mm; DV=−6.0 mm); slowly inserted the needle into the localized area of SN and Str, waited for 10 min, and then administered the drug of 6-OHDA at a rate of 0.4 L/min for 10 min, and each animal was given 4 L of each of SN and Str area, and then the needle was slowly withdrawn after the drug was administered for 10 min, and the PD rat model was established. Information regarding the modeling method of surgical procedures PD can also be found in literature which showed the injection site at localized area of SN and Str respectively (Sokoudi, et al. 2022 Physiol. Res. 71(4): 551-560; Haddadi, et al. 2013 Neuroscience Letters 555, 106-111).

### Testing Method

### 1. Apomorphine—Asymmetric Rotation Test

The animals were tested three weeks post-administration. 0.5 mg/kg of apomorphine was injected intraperitoneally. The number of rotations of each rat in 30 min were recorded, and the rat appeared to rotate in place toward the opposite side of the injury using the rotating forelimb as a support point. Information regarding apomorphine—asymmetric rotation test can also be found in the literature (Ximenes, et al. 2015 J Neurodegener Dis. 2015:313702).

### 2. Balance Beam Test

The rat was placed on a balance beam. The time was recorded it took for the rat to pass the balance beam as well as the number of foot slips (feet leave the top of the balance beam). The average of the two successful passing times and the number of foot slips were used as the evaluation criteria for the rat's locomotor balance ability. Information regarding beam test can also be found in the literature (Allbutt, et al. 2007 J Neurosci Methods 159(2):195-202; Fine, et al. 2014 Brain Res. 1574:96-104).

### 3. Grip Strength Test

The animal was placed on the platform with both forelimbs on the gripping pole, followed by grabbing the animal's tail, and pulling it straight back until the pulling force exceeded their gripping force. After the animals lose their grip, the preamplifier automatically recorded the maximum value of the pulling force. After the measurement was completed, the average of the maximum pulling force of each group of animals was calculated to evaluate the rat's locomotor muscle ability. Information on the grip strength test can also be found in the literature (Jeyasingham, et al. 2001 Brain Res Bull. 55(4):541-8).

### 4. Rotarod Test

The rotation speed of the rotary rod fatigue meter was set to 20 rpm/min with the test time of 5 min. The animals were placed on the rotating rod in batches for testing, and the time each animal spent on the rod was recorded to evaluate the motor coordination ability of the rats. Information on the rotarod test can also be found in the literature (Bohlen, et al. 2009 J Neurosci Methods 178(1):10-4).

### 6. Perfusion-Fixation-Sinking Sugar-Slice (Str/SN Brain Region)

After the rat was anesthetized, the abdominal cavity and thorax were cut open with straight scissors to expose the heart. The blood was first flushed out with normal saline perfusion, and then paraformaldehyde was used for initial perfusion fixation. The head was cut off, and the brain case was carefully opened with tweezers, and the brain tissue was removed and placed in paraformaldehyde solution and fix for 24 h; on the second day, took out the brain tissue and placed in 20% sucrose solution for 24 h; on the third day, took out the brain tissue and placed in 30% sucrose solution for 24 h; on the fourth day, took out the brain tissue and placed in 35% sucrose solution for 24 h (increase the time or concentration appropriately according to the condition of sugar deposition in the brain tissues); took out the brain tissue, embedded it in OCT embedding agent, and cut brain slices at the thickness of 16 m with a freezing microtome.

### 7. Immunofluorescence Staining (TH, Iba-1)

Immunohistochemical staining of TH and Iba-1 was performed on the sections. Sections were rewarmed for 30 min, closed at room temperature for 1 h with 50% serum+0.3% TritonX-100 in 50 μL per slice, shook off the slices, added primary antibody (diluted in PBS), overnight at 4° C.; rewarmed for 30 min; washed off primary antibody (PBS), 5 min×3 times; added secondary antibody (diluted in PBS) by backlighting, room temperature for 2 h; washed off secondary antibody (PBS), 5minx 3 times; added (4′,6-diamidino-2-phenylindole) (DAPI), and stayed at room temperature for 10 min; washed off DAPI, 5 min×3 times; added 70% glycerol to seal the slices to avoid air bubbles. Observed and took pictures under a microscope. Selected basically the same target brain area for each slice. Afterwards, ImageJ was used to count the signal number or area of the entire slice.

### 8. Elisa Test (IL-6, IL-1p, TNF-α)

The cerebrospinal fluid was directly extracted by inserting a needle through the foramen magnum, and the supernatant was collected after centrifugation for ELISA testing. The kit brand was Shanghai Enzyme Link. The product codes of IL-6, IL-1β, and TNF-α were m1064292, m1037361, and m1002859, respectively. Processed the sample according to the instructions of the ELISA detection kit, adjusted the blank well to zero, measured the absorbance (OD value) of each well sequentially at a wavelength of 450 nm, and calculated the sample concentration based on the standard curve and OD value.

### 9. Statistics

SPSS software was used for statistics analysis (one-way analysis of variance, p<0.05 is considered to have a significant difference), and Graph Pad software was used to draw images based on the SPSS analysis results. Adobe Photoshop software was used to generate the histological examination results. Extremely significant difference (p<0.001); significant difference (p<0.01); statistically significant difference (p<0.05).

### Example 2. Tolerability and Efficacy Test Using 6-OHDA Injection Induced Rat Model of Parkinson's Disease (PD)

Animal Model: PD model is established via stereotactic injection of 6-OHDA into Sprague-Dawley rats' brain.

### 1. Methods:

In this study, 16 Sprague Dawley (SD) male rats 180g-220g were used to establish a PD model by stereotactic unilateral injection of 6-OHDA in the substantia nigra (SN) and striatum (Str) brain areas after one week of acclimatization feeding and were randomly divided into 2 groups of 8 rats each. One group was dosed with the test compound BTUDC 500 mg/kg (QD, p.o.) and the other group was the Model group, which was given an equal volume of 0.5% CMC-Na (QD, p.o.) as the test compound group. The animals were administered once a day for 21 consecutive days starting from the second day after modeling to evaluate the tolerability and therapeutic potential of BTUDC in the 6-OHDA PD model.

### 2. General Condition Observation:

The body weight and food intake of the rats were collected and recorded every day, and the cage side observation for the animals was carried out.

### 3. Behavioral Tests:

After 21 days of administration, the following tests were conducted: apomorphine-asymmetric rotation test, balance beam test, grip strength test and rotarod test.

### 4. Results:

During the test period, the animals in each group were in good condition, with no abnormalities or accidental deaths. Both the drug administration group and the Model group showed weight loss on D1 after modeling due to surgical trauma, and the weight increased steadily from D2 after gradual recovery from the trauma, and the body weight change was similar between the two groups. Food intake increased steadily in four days after modeling in both the drug-administered group and the Model group, and stabilized from D5 onwards.

### Tolerability and Efficacy:

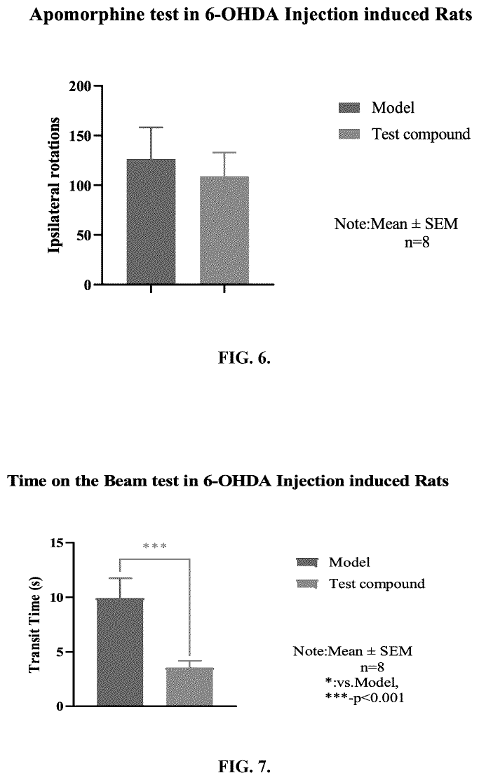

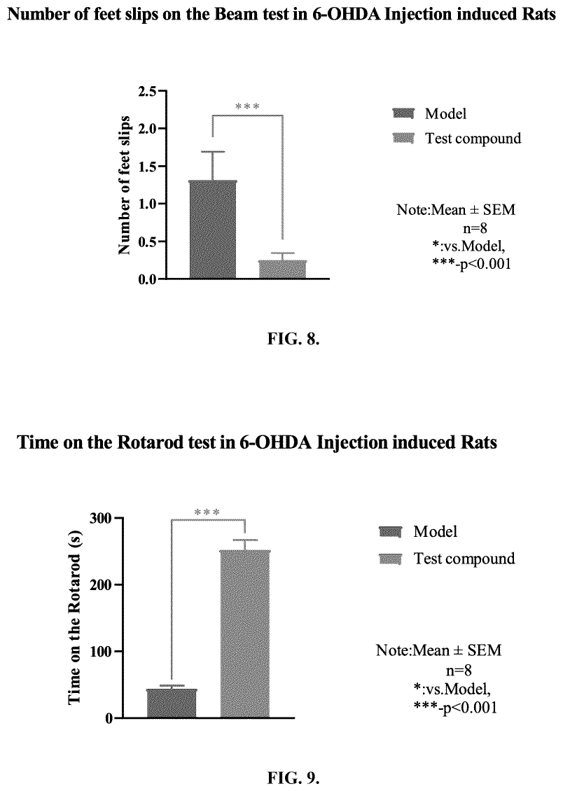

FIGS. 6-10 shows exemplary results of various experiments involving ipsilateral rotation, time on a beam, feet slipping on a beam, time on rotarod, and grip strength tests.

On the 21st day after stereotaxic injection of 6-OHDA, all rats showed in situ rotation behavior with the rotating forelimb as the support point to the injured contralateral side after Apomorphine (APO) induction, indicating that the model was successfully established. There was no statistically significant difference in number of ipsilateral rotations within 30 minutes between the Test Compound groups and Model groups (p>0.05).

TABLE 7
Effect of Test Compound on Number of Ipsilateral Rotation within
30 Minutes of Apomorphine Injection after 21 days treatment.

| | | Number of Ipsilateral |
|---|---|---|
| Group | Dosage | Rotation |
| Model | Vehicle | 126.380 ± 31.681 |
| Test compound | 500 mg/kg/day | 109.000 ± 23.670 |

There were no obvious differences between the model control group and the Test Compound treatment group during the whole study period for the body weight and food intake data. In addition, no Test Compound-related mortality or moribundity nor abnormal animal state was noted in the Test Compound treated group as compared with model group, which indicated that the Test Compound was well tolerated in the PD rat model. Compared with the Model group, after 21 days of continuous treatment with the Test Compound, the rats in the Test Compound group showed a significant decrease in the passing time (p<0.001) and the number of feet slips (p<0.001) in the balance beam test, a significant increase in the rod time in the rotarod test (p<0.001), and a significant increase in the peak grip strength in the grip strength test (p<0.001).

TABLE 8
Behavioral Examination in 6-OHDA Stereotactic Injection Induced PD Rats after
21 Days Compound Treatment (Beam Test, Rotarod Test, Grip Strength Test)

| | | | | Rotarod | Grip Strength |
|---|---|---|---|---|---|
| | | Beam Test | | Test | Test |
| | | Passing | Number of feet | Rod | Peak grip |
| Group | Dose | time(s) | slips | time(s) | strength(gf) |
| Model | Vehicle | 9.938 ± 1.8088 | 1.313 ± 0.3772 | 44.063 ± 4.6049 | 535.521 ± 15.5632 |
| Test | 500 mg/kg | 3.563 ± 0.6228 *** | 0.250 ± 0.0945 *** | 252.413 ± 14.4032 *** | 757.479 ± 7.5044 *** |
| Compound | | | | | |

Note:
As compared with the Model group,
*** P < 0.001

This study showed that PD rat model was well tolerated to the Test Compound and the Test Compound showed beneficial effects to Parkinson's Disease model animals which were induced by stereotactic injection of 6-OHDA into rats' substantia nigra and striatum.

### Example 3. Pharmacodynamic Study for Parkinson's Disease

Animal Model: PD model is established via stereotactic injection of 6-OHDA into Sprague-Dawley rats' brain.

Experimental Methods: Seventy 180-220g SD male rats were selected and fed for one week for accumulation, and the PD model was established by stereotactic unilateral injection of 6-OHDA in the Substantia Nigra (SN) and Striatum (Str) regions and were randomly divided into five groups according to their body weights including Madopar group (Madopar, 50 mg/kg), BTUDC-L group (BTUDC, 100 mg/kg), BTUDC-M group (BTUDC, 500 mg/kg), and BTUDC-H group (BTUDC, 1000 mg/kg), with 10 animals in each group. Another 10 SD male rats were taken for sham operation (only 4 μL of 0.9% NS containing 0.02% ascorbic acid was given to the SN and Str brain regions during modeling) as the Sham group. The drug was administered by gavage once daily continuously for 21 days starting on the second day of modeling (first day of dosing, D1). General observation was made during the dosing period. At the end of the administration, the test articles were evaluated for tolerance in this model and in behavioral apomorphine—asymmetric rotation test, beam test, rotarod test and grip strength test, immunofluorescence staining (TH, Ibal), and cerebrospinal fluid inflammatory factor assays to evaluate the pharmacodynamic effects of the test articles. Inflammatory factors tested included interleukin 6 (1L-6), inflammatory factor Interleukin 1j (1L-10), Tumor Necrosis Factor alpha (TNF-α), inflammatory assays were done in a total of 4 groups included Sham, Model, Madopar and BTUDC-H groups.

TABLE 9

| | | | | | Dose | Conc'n | | Frequency and |
|---|---|---|---|---|---|---|---|---|
| No. | Group | Number | Model | Drug | (mg/kg) | (mg/mL) | Route | periodicity |
| G1 | Sham | 10 | Sham | 0.5% CMC- | — | — | p.o. | Qd, D 1-D 21 |
| | | | | Na | | | | |
| G2 | Model | 10 | 6-OHDA | 0.5% CMC- | — | — | p.o. | Qd, D 1-D 21 |
| | | | model | Na | | | | |
| G3 | Madopar | 10 | 6-OHDA | levodopa + | 50 | 5 | p.o. | Qd, D 1-D 21 |
| | | | model | benserazide | | | | |
| G4 | BTUDC_L | 10 | 6-OHDA | BTUDC | 100 | 10 | p.o. | Qd, D 1-D 21 |
| | | | model | | | | | |
| G5 | BTUDC_M | 10 | 6-OHDA | BTUDC | 500 | 50 | p.o. | Qd, D 1-D 21 |
| | | | model | | | | | |
| G6 | BTUDC_H | 10 | 6-OHDA | BTUDC | 1000 | 100 | p.o. | Qd, D 1-D 21 |
| | | | model | | | | | |

Dosing volume: 10 mL/kg
1. General observation: During the period of drug administration, body weight, food intake test and cage side observation were conducted every 3 days.
2. Behavioral test: After 21 days of administration, behavioral test was carried out on the following day.
3. Histochemical test: After the behavioral test, the cerebrospinal fluid from each animal (N=10) was collected, centrifuged to collect the supernatant, and stored at −80° C. for Elisa test (IL-6, IL-1β, TNF-α), then the animals in each group (N=5) were randomly selected to take the modeling side of the striatum for the detection of neurotransmitters, and the rest of them (N=5) were subjected to cardiac perfusion, which was performed by the process of perfusion, fixation, sedimentation and slicing for immunofluorescence staining and photographed, and the number or area of signals were counted by ImageJ software.
4. Data analysis: After the data were summarized and counted, they were analyzed using SPSS data statistics software (one-way ANOVA, p<0.05 was considered a significant difference), and images were plotted according to the results of SPSS analysis using Graph Pad software. Adobe Photoshop software was used to generate the histological examination results.

### Results

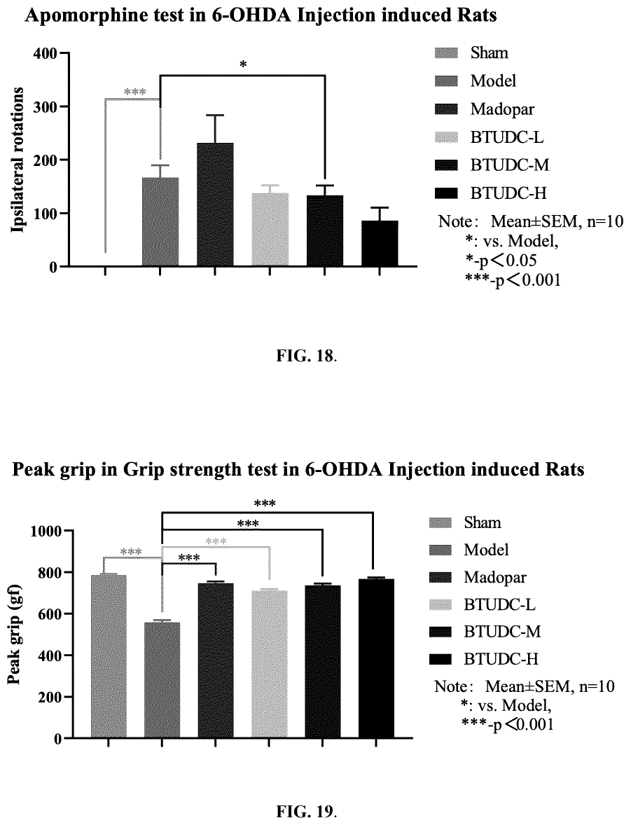

The rotation test results of APO-induced PD model rats are shown in FIG. 18. After APO induction, the rats in each administration group and the Model group showed the behavior of turning in place toward the opposite side of the injury using the rotating forelimb as a support point. Compared with the Sham group, the rats in the Model group rotated in circles within 30 minutes after apomorphine injection. The number of rotations increased significantly (p<0.001), indicating that the model was successfully established. Compared with the Model group, BTUDC-H group rats with apomorphine injection had significantly lower number of rotational circles within 30 min, indicating that BTUDC has the potential to protect neurons in the brain.

TABLE 10
Results of Behavioral test (Grip strength, Rotarod and Beam) in PD Model Rats

| | Dose | Peak Grip | Time on the | Passage | Foot Slips |
|---|---|---|---|---|---|
| Groups | (mpk) | Strength | Rod (s) | Time (s) | Times |
| Sham | — | 786.800 ± 4.499*** | 299.315 ± 0.679*** | 3.7 ± 0.3*** | 0.2 ± 0.1*** |
| Model | — | 558.334 ± 10.175 | 63.250 ± 9.871 | 10.9 ± 1.3 | 1.7 ± 0.2 |
| Madopar | 50 | 746.483 ± 8.607*** | 185.370 ± 22.103*** | 6.9 ± 0.6*** | 0.7 ± 0.2*** |
| BTUDC-L | 100 | 711.616 ± 7.934*** | 103.220 ± 5.806 | 8.0 ± 0.7** | 1.0 ± 0.3* |
| BTUDC-M | 500 | 737.000 ± 8.222*** | 148.070 ± 21.760*** | 6.4 ± 0.7*** | 0.8 ± 0.2** |
| BTUDC-H | 1000 | 767.101 ± 6.668*** | 172.625 ± 17.355*** | 4.8 ± 0.4*** | 0.6 ± 0.2*** |

Note:
Mean ± SEM, N = 10;
***p < 0.001 vs. Model;
**p < 0.01 vs Model;
*p < 0.05 vs. Model.

The results of the grip strength test of PD model rats are shown in FIG. 19 and Table 10. In comparison, the peak grip strength of rats in each administration group (G3-G6) was significantly higher than that of the Model group, and the difference was extremely significant (p<0.001). Different doses of BTUDC administration groups improved the grip strength in a dose-dependent manner.

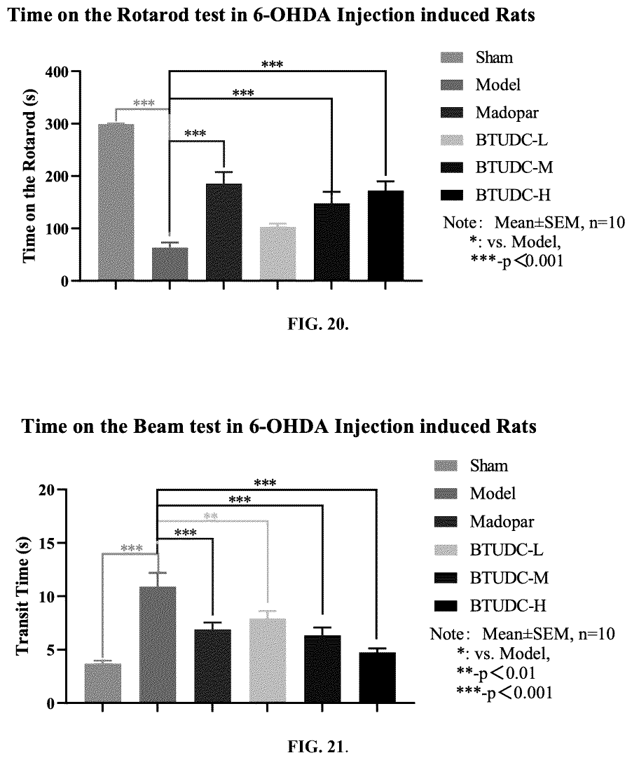

The results of the rotarod test of PD rats are shown in FIG. 20. The time on the rod of rats in each administration group (G3-G6) was significantly higher than that of the Model group; the effect of different doses of BTUDC administration groups on the improvement of the time on the rod was dose-dependent, among which the on-rod time of the mid-dose and high-dose BTUDC groups was statistically higher than that of the Model group (p<0.001).

The results of the time for PD rats to pass the balance beam in the balance beam test are shown in FIG. 21. Compared with the Model group, the time for rats in each administration group (G3-G6) to pass the balance beam was significantly shorter than that of the Model group (p<0.01); the improvement effect of different doses of BTUDC on the time to pass the balance beam was dose-dependent.

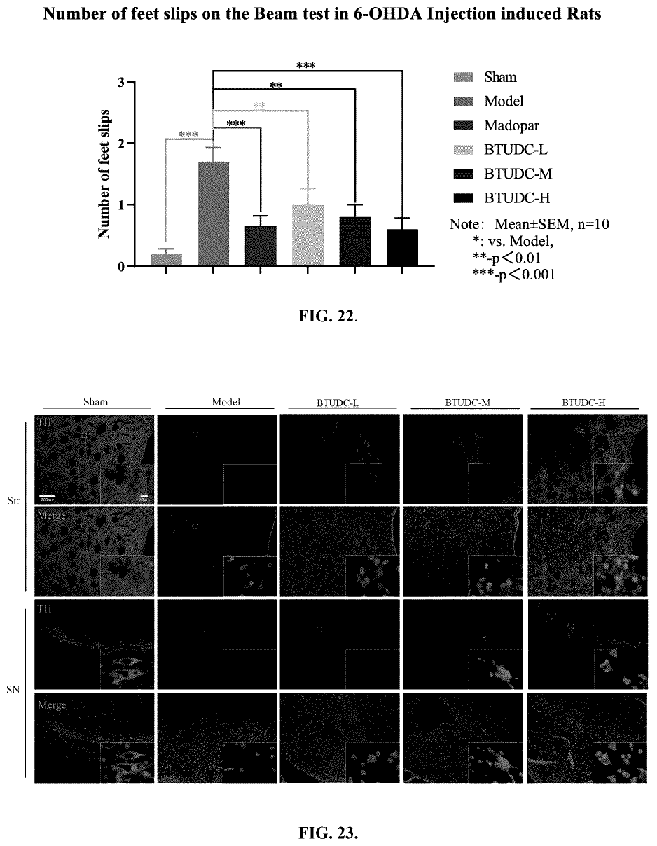

The results of the number of foot slips of PD rats in the beam test are shown in FIG. 22. Compared with the Model group, the number of times rats in each administration group (G3-G6) slipped on the balance beam was significantly less than the Model group (p<0.05); the improvement effect of different doses of BTUDC on the number of times the rats slipped on the balance beam was dose-dependent.

TABLE 11
Result of TH staining and Iba-1 staining in Str and SN brain region

| | | TH staining | | Iba-1 staining | |
|---|---|---|---|---|---|
| | | Str | SN | Str | SN |
| | | Fluorescence | positive | positive | positive |
| Groups | dose (mpk) | intensity | cells num. | cells num. | cells num. |
| Sham | — | 117.0 ± 2.1*** | 58.9 ± 3.8*** | 85.4 ± 9.8*** | 30.7 ± 3.9*** |
| Model | — | 11.1 ± 3.0 | 0.9 ± 0.5 | 668.3 ± 13.0 | 397.8 ± 21.2 |
| BTUDC-L | 100 | 19.8 ± 3.5 | 0.7 ± 0.3 | 601.7 ± 14.2** | 413.7 ± 42.8 |
| BTUDC-M | 500 | 32.6 ± 5.5*** | 2.0 ± 0.6 | 602.7 ± 9.1** | 393.1 ± 20.1 |
| BTUDC-H | 1000 | 65.8 ± 4.4*** | 12.7 ± 3.1*** | 556.5 ± 16.2*** | 270.0 ± 21.5*** |

Note:
Mean ± SEM, N = 15;
***p < 0.001 vs. Model;
**p < 0.01 vs Model;
*p < 0.05 vs. Model;
5 animals per group;
Each animal was sectioned three times for TH staining and Iba-1 staining statistics, N = 5*3.

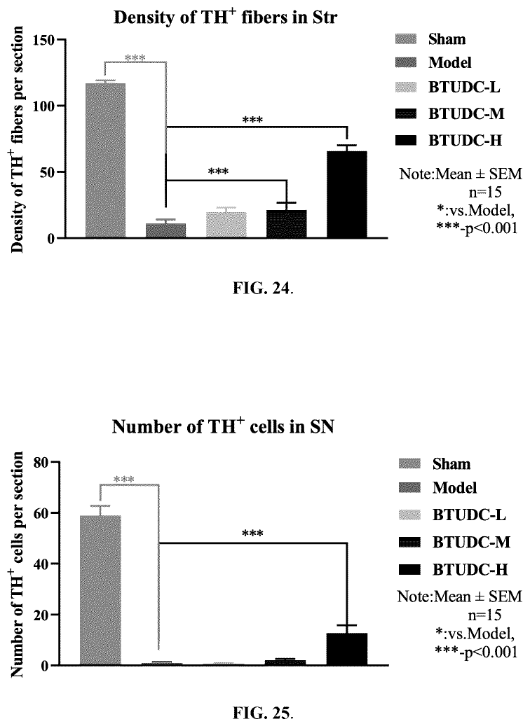

The results of TH staining are shown in FIG. 23. The TH staining results of fluorescence intensity in the Str brain area of rats after 21 days of administration are shown in Table 11 and FIG. 24. The TH staining results of the number of positive cells in the SN brain area of rats after 21 days of administration are shown in Table 11 and FIG. 25. Compared with the Sham group, the TH staining of fluorescence intensity in the Str brain area and the number of TH staining positive cells in the SN brain area were significantly lower in the Model group rats, and the difference was extremely significant (p<0.001). Compared with the Model group, the TH staining fluorescence intensity of the rat Str brain area in each administration group (G4-G6) was significantly higher than that of the Model group (p<0.05), and the number of TH-positive cells in the SN brain area of the BTUDC-H group was significantly higher than that of the Model group (p<0.001). The improvement effect of BTUDC on TH fluorescence intensity in the Str brain area and the number of TH-positive cells in the SN brain area of 6-OHDA rats was dose-related. It is suggested that BTUDC has beneficial effects in improving the substantia nigra lesions and has neuronal protective effects.

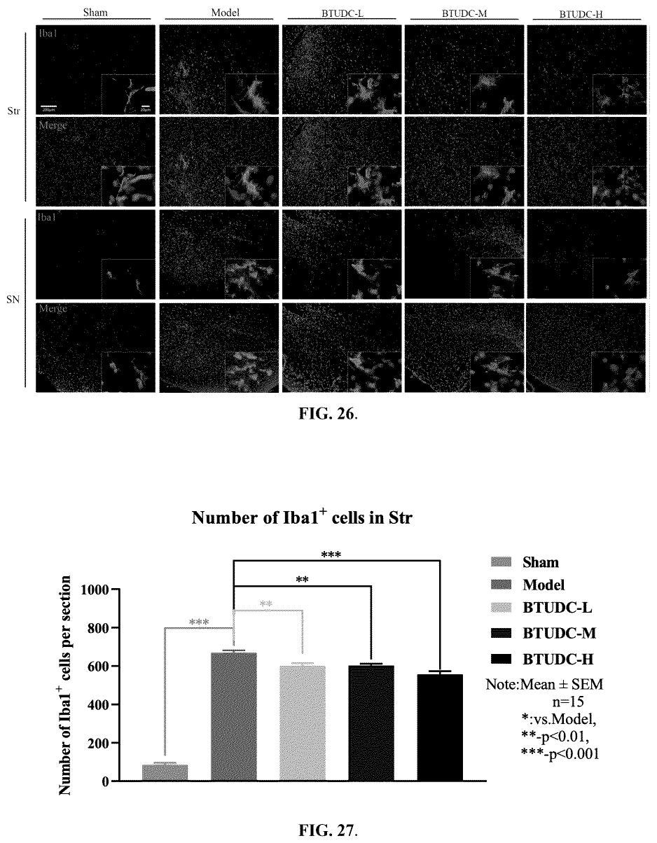

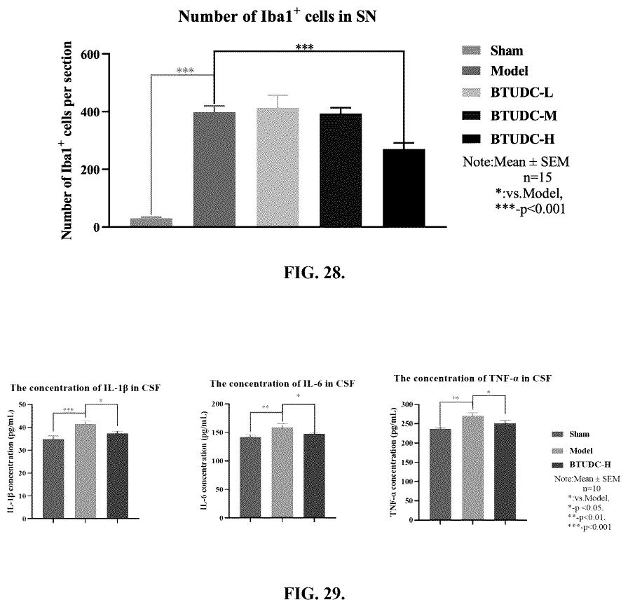

The Iba-1 staining results are shown in FIG. 26. The Iba-1 staining results for the number of positive cells in the Str brain area of the rats after 21 days of administration are shown in FIG. 27. The results of the number of positive cells are shown in FIG. 28. Compared with the Model group, the number of Ibal-positive cells in the Str brain area of rats in each administration group (G4-G6) was significantly lower than that in the Model group (p<0.05). The number of Ibal-positive cells in the SN brain area of the BTUDC-H group was significantly lower than the Model group (p<0.001). The improvement effect of BTUDC on the number of Ibal-positive cells in the Str brain area and SN brain area of 6-OHDA rats showed a certain dose correlation, indicating that BTUDC has the potential to improve chronic neuroinflammation and has neuroprotection effects.

TABLE 12
Elisa test results of 1L-1β/IL-6/TNF-α in rat cerebrospinal
fluid after 21 days of administration

| | Dose | IL-1β | IL-6 | TNF-α |
|---|---|---|---|---|
| Groups | (mpk) | Conc. (pg/mL) | Conc. (pg/mL) | Conc. (pg/mL) |
| Sham | — | 34.966 ± 1.311*** | 142.124 ± 2.970** | 236.978 ± 4.435** |
| Model | — | 41.509 ± 1.298 | 159.058 ± 6.349 | 271.022 ± 8.145 |
| BTUDC-H | 1000 | 37.251 ± 1.005* | 147.095 ± 1.871* | 250.424 ± 9.186* |

Note:
Mean ± SEM, n = 10;
*p < 0.05,
**p < 0.01,
***p < 0.001 vs. Model

The Elisa test results of 1L-1β in rat cerebrospinal fluid after administration for 21 days are shown in Table 12 and FIG. 29.

Elisa test results of 1L-6 in rat cerebrospinal fluid after 21 days of administration are shown in Table 12 and FIG. 30.

Analysis of elisa test results of TNF-α in rat cerebrospinal fluid after 21 days of administration is shown in FIG. 31.

In summary, BTUDC was well tolerated by the animals up to 1000 mg/kg, and there are no obvious abnormalities in body weight growth or food intake compared with other groups of animals. BTUDC showed beneficial effects in various behavioral tests such as grip strength, rotarod, and balance beam. The beneficial effects were dose-dependent with the improvement effects in the medium and high dose groups achieved statistical significance. In addition, BTUDC also increased the number and intensity of tyrosine hydroxylase-positive cells and reduced the number of microglia. BTUDC dose-dependently improved the fluorescence intensity of tyrosine hydroxylase (TH)-positive cells in the Str brain area. The high-dose BTUDC group significantly increased the number of TH-positive cells in the SN brain area. All the BTUDC treatment groups significantly reduced the number of microglia (Ibal) in the Str brain area. The high-dose BTUDC group significantly reduced the number of microglia (Ibal) in the SN brain area.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

1. A salt having Formula (I):

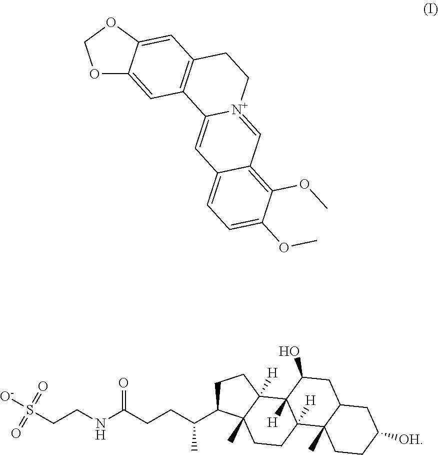

2. A salt in substantially pure form, wherein the salt is represented by Formula (I):

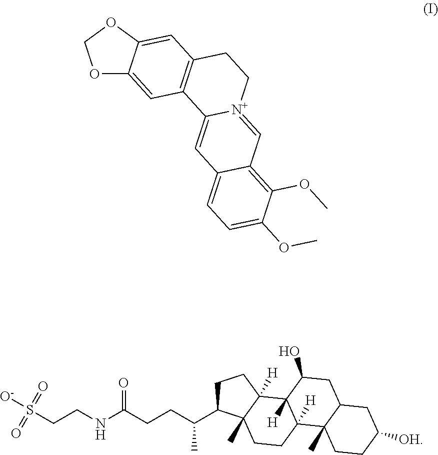

3. The salt of claim 2, wherein the salt is characterized by a purity equal to or greater than 95%.

4. The salt of claim 1, wherein the salt is made by an acid-base reaction between berberine (BBR) and tauroursodeoxycholic acid (TUDCA).

5. A solid form of a compound of Formula (I), which is Form Å, wherein X-ray powder diffraction (XRPD) pattern thereof comprises one or more characteristic diffraction peaks at the following 2θ angles: 4.62°, 9.32°,17.02°±0.2°, with a radiation source of Cu-Kα.

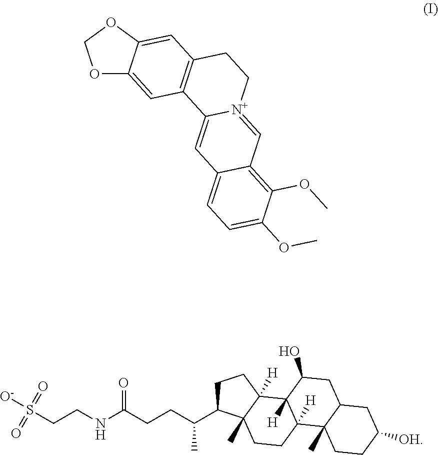

6. The solid form according to claim 5, wherein the XRPD pattern of the solid form further comprises one or more characteristic diffraction peaks at the following 20 angles: 5.96°, 6.23°, 15.19°+0.2°, with a radiation source of Cu-Kα.

7. The solid form according to claim 5, wherein the XRPD pattern of the solid form comprises characteristic diffraction peaks at the following 2θ angles: 4.62°, 5.96°, 6.23°, 9.32°, 15.19°, 17.020±0.2°, with a radiation source of Cu-Kα.

8. The solid form according to claim 7, wherein the XRPD pattern of the solid form further comprises one or more than one of characteristic diffraction peaks at the following 2θ angles: 11.99°,12.56°,12.900±0.2°, with a radiation source of Cu-Kα.

9. The solid form according to claim 5, wherein the XRPD pattern of the solid form comprises characteristic diffraction peaks at the following 2θ angles: 4.62°, 5.96°, 6.23°, 9.32°, 11.99°, 12.56°, 12.90°, 13.32°, 14.25°, 14.88°, 15.19°, 17.02°, 17.51°, 17.73°, 18.02°,21.39°, 24.25°, 24.71°+0.2°, with a radiation source of Cu-Kα.

10. The solid form according to claim 5, wherein a differential scanning calorimetry (DSC) curve of the solid form comprises an endothermic peak with a peak value at about 280° C.

11. The solid form according to claim 5, wherein a thermogravimetric analysis (TGA) curve of the solid form comprises a weight loss of about 0.5% to about 3% from room temperature to 150° C.

12. The solid form according to claim 5, wherein the solid form is characterized by a BTUDC:H₂O ration of 1:X, wherein X is in a number in the range of 0 to 2.

13. The solid form of claim 12, wherein the solid form is anhydrate.

14. The solid form of claim 12, wherein the solid form is hydrate with up to 2 H₂O molecules per BTUDC molecule.

15. The solid form of claim 5, wherein the solid form is a crystalline form.

16. A pharmaceutical composition comprising the salt of claim 1, and a pharmaceutically acceptable excipient, carrier, or diluent.

17. A pharmaceutical composition comprising the solid form of claim 5, and a pharmaceutically acceptable excipient, carrier, or diluent.

18. A unit dosage for comprising the pharmaceutical composition of claim 16.

19. A method for reducing, preventing or treating a neurodegenerative disease, comprising administering to a subject in need thereof a pharmaceutical composition comprising the salt of claim 1.

20. A method for reducing, preventing or treating a neurodegenerative disease, comprising administering to a subject in need thereof a pharmaceutical composition comprising the solid form of claim 5.

21-51. (canceled)
